(19) United States
(12) Patent Application Publication
(10) Pub. No.: US 2023/0375552 A1
(43) Pub. Date: Nov. 23, 2023

(54) INTEGRATED PROTEOMIC BIOMARKERS FOR THE DETECTION OF AGGRESSIVE PROSTATE CANCER

(71) Applicant: THE JOHNS HOPKINS UNIVERSITY, Baltimore, MD (US)

(72) Inventors: Daniel Wan-Yui Chan, Clarksville, MD (US); Zhen Zhang, Baltimore, MD (US); Lori Jean Sokoll, Owings Mills, MD (US); Jin Song, Clarksville, MD (US); Shiyong Ma, Baltimore, MD (US); Hui Zhang, Ellicott City, MD (US)

(21) Appl. No.: 18/247,144

(22) PCT Filed: Sep. 29, 2021
(86) PCT No.: PCT/US2021/052623

§ 371 Date: Mar. 29, 2023

Related U.S. Application Data
(60) Provisional application No. 63/084,798, filed on Sep. 29, 2020.

(51) Int. Cl. G01N 33/574

(52) U.S. Cl. CPC G01N 33/57434; G01N 2440/38; G01N 2333/96455

(57) ABSTRACT

The present invention provides compositions and methods useful for detecting and treating aggressive prostate cancer. In a specific embodiment, a method for identifying a patient as having aggressive prostate cancer comprises the steps of (a) measuring the concentration of total PSA, free PSA, p2PSA in a serum sample obtained from the patient and calculating phi based on the measured serum concentrations; (b) measuring the concentration of fucosylated PSA (fuc-PSA) in a serum sample obtained from the patient; (c) measuring the concentration in a serum sample obtained from the patient of one or more of the following biomarkers: B7-H3, PLA2G7, GDF-15, IL-6R alpha, SDC1, VCAM-1, s Tie-2, IL-16, CA15-3, MMP-2, and H SP27; and (d) using an algorithm to identify the patient as having aggressive prostate cancer based on a panel of biomarkers comprising phi, fuc-PSA and one or more of the serum concentrations measured in step (c).

### CROSS-REFERENCE TO RELATED APPLICATIONS

This application claims the benefit of U.S. Provisional Application No. 63/084,798, filed Sep. 29, 2020, which is incorporated herein by reference in its entirety.

### GOVERNMENT SUPPORT CLAUSE

This invention was made with government support under grant no. CA115102, awarded by the National Institutes of Health. The government has certain rights in the invention.

### FIELD OF THE INVENTION

The present invention relates to the field of cancer. More specifically, the present invention provides compositions and methods useful for detecting and treating aggressive prostate cancer.

### BACKGROUND OF THE INVENTION

Prostate cancer (PCa) is the most common non-cutaneous solid tumor in men and has a high prevalence among men aged 50 years and above in the United States. In 2021, new cases are estimated at 248,530 with approximately 34,130 deaths [1]. The serum test for prostate-specific antigen (PSA) was developed and approved by the Food and Drug Administration (FDA) for prostate cancer over 30 years ago [2]. While PSA has become a routine clinical test, PSA screening has garnered substantial criticism in recent years due to the potential for overdetection and overtreatment of PCa. In particular, recommendations by the United States Preventive Services Task Force (USPSTF) [3] have generated significant debate regarding PSA-based screening. Biopsies trigged by a marginally elevated serum PSA level or other reason will likely result in a significant number of biopsy-positive cases for whom the majority will have low risk disease that may not require active clinical intervention. Overtreatment could be mitigated with a diagnostic test capable of identifying aggressive (AG) PCa prior to biopsy. While there is no consensus on the definition of “aggressiveness,” it is generally agreed that Gleason score (GS) is likely the best indicator. In general, higher GSs are associated with more aggressive PCa defined in terms of disease-free survival [4, 5]. The most widely accepted histological cutoff for PCa is GS 7. When the GS is 7 or higher, the tumor is considered “aggressive”. There is a great need for the development of diagnostics for the detection and treatment of AG PCa.

### SUMMARY OF THE INVENTION

Accordingly, in one aspect, the present invention provides compositions and methods for identifying a patient as having aggressive prostate cancer comprising the steps of (a) measuring the concentration of total PSA, free PSA, p2PSA in a serum sample obtained from the patient and calculating phi based on the measured serum concentrations; (b) measuring the concentration of fucosylated PSA (fuc-PSA) in a serum sample obtained from the patient; (c) measuring the concentration in a serum sample obtained from the patient of one or more of the following biomarkers: B7-H3, PLA2G7, GDF-15, IL-6 R alpha, SDC1, VCAM-1, sTie-2, IL-16, CA15-3, MMP-2, and HSP27; and (d) using an algorithm to identify the patient as having aggressive prostate cancer based on a panel of biomarkers comprising phi, fuc-PSA and one or more of the serum concentrations measured in step (c).

In one embodiment, the panel of step (d) comprises phi, fuc-PSA, SDC1 and GDF-15. In another embodiment, the panel of step (d) comprises phi, fuc-PSA, SDC1 and Tie-2.

In particular embodiments, measurement steps (a) and (c) are performed using an immunoassay. In other embodiments, measurement step (b) is performed using a lectin assay followed by an immunoassay. In certain embodiments, the method further comprises the step of treating the patient with a prostate cancer therapy. In particular embodiments, the prostate cancer therapy comprises prostatectomy, radiation therapy, cryotherapy, hormone therapy, chemotherapy, immunotherapy and combinations thereof. The present invention can be used as part of an active surveillance program of monitoring prostate cancer.

In certain embodiments, the panel of step (d) further comprises PSA and % fuc-PSA. In a specific embodiment, the panel comprises phi, fuc-PSA, SDC1, GDF-15, IL-6 R alpha, MMP-2 and CA15-3. In another specific embodiment, the panel comprises phi, fuc-PSA and PLA2G7. In a further embodiment, the panel comprises phi, fuc-PSA, PSA, % fuc-PSA and GDF-15.

In yet another embodiment, the panel comprises phi, fuc-PSA, PSA, % fuc-PSA and B7-H3. In an alternative embodiment, the panel comprises phi, fuc-PSA, PSA, % fuc-PSA, GDF-15, SDC1, Tie-2 and VCAM-1. In another specific embodiment, the panel comprises phi, fuc-PSA, PSA, % fuc-PSA, GDF-15, B7-H3, Tie-2, and SDC1.

In another specific embodiment, a method for identifying a patient as having aggressive prostate cancer comprises the steps of (a) measuring the concentration of total PSA, free PSA, p2PSA in a serum sample obtained from the patient and calculating phi based on the measured serum concentrations; (b) measuring the concentration of fucosylated PSA (fuc-PSA) in a serum sample obtained from the patient; and (c) using an algorithm to identify the patient as having aggressive prostate cancer based on a panel of biomarkers comprising phi and fuc-PSA.

In a more specific embodiment, measurement step (a) is performed using an immunoassay. In another specific embodiment, measurement step (b) is performed using a lectin assay followed by an immunoassay.

In particular embodiments, the method further comprises the step of treating the patient with a prostate cancer therapy. The prostate cancer therapy can comprise prostatectomy, radiation therapy, cryotherapy, hormone therapy, chemotherapy, immunotherapy and combinations thereof.

In another aspect, the present invention provides compositions and methods for treating a patient having aggressive prostate cancer. In one embodiment, a method for treating a patient having aggressive prostate cancer comprises the step of administering a prostate cancer therapy to a patient identified as having aggressive prostate cancer using a method described herein.

### BRIEF DESCRIPTION OF THE FIGURES

FIG. 1A-10. Analysis of biomarkers in sera from NAG (low risk/non-aggressive) and AG PCa patients as well as biopsy negative controls. FIG. 1A-10: B7-H3, PLA2G7, GDF-15, IL-6 R alpha, SDC1, VCAM-1, Tie-2, IL-16, CA15-3, MMP-2, HSP27, Fuc-PSA, PSA, % fPSA, and phi in NAG and AG PCa patients as well as biopsy negative controls (non-PCa) are demonstrated in overlaid scatterplots and boxplots. Only biomarkers demonstrating significant differences between AG and NAG PCa (or between AG and NAG+non-PCa) are shown with asterisks (Mann-Whitney U test). Bars in the boxes, median value. *, P<0.05; **, P<0.01; ***, P<0.001; ****, P<0.0001.

FIG. 2A-2B. Univariate evaluation of serum biomarkers. Label permutation and bootstrap methods were used to evaluate statistical stability of the diagnostic performance of individual biomarkers in separating AG from NAG (low risk/non-aggressive) PCa(FIG. 2A) or NAG PCa and non-PCa (FIG. 2B). AUC means (95% CI) and STDs are presented.

FIG. 3A-3B. Multivariate evaluation of serum biomarkers. Diagnostic performance of combined serum biomarkers in separating AG from NAG (low risk/non-aggressive) PCa (FIG. 3A) or NAG PCa and non-PCa (FIG. 3B). ROC curves with AUCs are presented.

FIG. 4A-4B. Batch effect identification and correction. FIG. 4A: Distributions of Fuc-PSA in the two batches (Runs 1, 2 and Runs 3, 4, 5). FIG. 4B: Normalized distributions of Fuc-PSA in the two batches.

FIG. 5A-5D. PCA biplot of individual serum biomarkers. PCA biplot combined with PCA score plot and loading plot was used to analyze individual serum biomarkers for classifying patients with AG and NAG (low risk/non-aggressive) PCa only (FIG. 5A & 5C) or classifying AG and NAG PCa patients as well as biopsy negative controls (FIG. 5B & 5D), demonstrating clusters of samples based on their similarity (PCA score plot) and how strongly each characteristic influences a principal component (i.e., PC-1 and PC-2).

FIG. 6A-6B. The scatterplot matrix of selected serum biomarkers. The complementarities of 9 selected serum biomarkers in separate AG either from NAG (low risk/non-aggressive) PCa only (FIG. 6A) or NAG PCa and non-PCa (FIG. 6B) are demonstrated.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, wood chucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is mouse or mice. In various embodiments, the subject, patient or individual is human.

A subject can be one who has been previously diagnosed with or identified as suffering from or having a condition, disease, or disorder in need of treatment (e.g., prostate cancer) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a disease, a subject that has a disease, a subject diagnosed with a disease, a subject that has been treated for a disease, a subject that is being treated for a disease, and a subject that is at risk of developing a disease.

In some embodiments, the subject is selected from the group consisting of a subject suspected of having prostate cancer, a subject that has prostate cancer, a subject diagnosed with prostate cancer, a subject that has non-aggressive prostate cancer, a subject suspected of having aggressive prostate cancer, a subject that has been treated for prostate cancer, a subject that is being treated for prostate cancer, and a subject that is at risk of developing prostate cancer.

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made. In certain embodiments, a subject can be at risk of developing aggressive prostate cancer.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a serum sample. In particular embodiments, the sample is a urine sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{1M}) samples can be stored and mailed, and an assay can be performed remotely.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function s in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “peptide” as used herein refers to any compound containing at least two amino acid residues joined by an amide bond formed from the carboxyl group of one amino acid residue and the amino group of the adjacent amino acid residue. In some embodiments, peptide refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In some embodiments, the peptide ranges in length from 2 to about 12 residues, or 2 to about 20 residues, or 2 to about 30 residues, or 2 to about 40 residues, or 2 to about 50 residues, or 2 to about 60 residues, or 2 to about 70 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. 11th American Peptide Symposium, ESCOM Science Publishers, The Netherlands, and the like)).

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide,” A protein contains at least one polypeptide. Short polypeptides, e.g., containing less than 20-30 residues, are sometimes referred to as “peptides.”

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy-chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the biomarkers are detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous when mere detection indicates the diagnosis. The term is also used synonymously with the term “measuring.”

The terms “marker” or “biomarker” are used interchangeably herein, and in the context of the present invention refer to a protein or peptide (for example, protein or peptide associated with prostate cancer or prostate cancer as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof.

A “test amount” of a marker refers to an amount of a marker present in a sample being tested. A test amount can be either in absolute amount (e.g., g/mL) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed, A control amount can be either in absolute amount (e.g., μg/ml) or a relative amount (e.g., relative intensity of signals).

The term “differentially present” or “change in level” refers to differences in the quantity and/or the frequency of a marker present in a sample taken from patients having a specific disease or disorder as compared to a control subject. For example, a marker can be present at an elevated level or at a decreased level in samples of patients with the disease or disorder compared to a control value (e.g., determined from samples of control subjects). Alternatively, a marker can be detected at a higher frequency or at a lower frequency in samples of patients compared to samples of control subjects. A marker can be differentially present in terms of quantity, frequency or both as well as a ratio of differences between two or more specific modified amino acid residues and/or the protein itself. In one embodiment, an increase in the ratio of modified to unmodified proteins and peptides described herein is diagnostic of any one or more of the diseases described herein. In particular embodiments, a marker can be differentially present in patients having aggressive prostate cancer as compared to a control subject including patients having non-aggressive prostate cancer or no cancer.

A marker, compound, composition or substance is differentially present in a sample if the amount of the marker, compound, composition or substance in the sample (a patient having aggressive prostate cancer) is statistically significantly different from the amount of the marker, compound, composition or substance in another sample (a patient having non-aggressive cancer or no cancer), or from a control value (e.g., an index or value representative of non-aggressive cancer or no cancer). For example, a compound is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or disorder, is statistically significantly higher or lower than in the control samples or control values obtained from controls such as a subject having non-aggressive prostate cancer, benign lesions and the like, or otherwise healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples (e.g., a patient having aggressive prostate cancer) than the other set of samples (e.g., a patient having non-aggressive prostate cancer or no cancer). These exemplary values notwithstanding, it is expected that a skilled practitioner can determine cut-off points, etc., that represent a statistically significant difference to determine whether the marker is differentially present.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemai, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

### II. Measurement/Detection of Markers

In one aspect, the present invention provides compositions and methods for measuring one or more proteins. In specific embodiments, the proteins comprises one or more of total PSA, free PSA, p2PSA, fuc-PSA, PSA, B7-H3, PLA2G7, GDF-15, IL-6 R alpha, SDC1, VCAM-1, sTie-2, IL-16, CA15-3, MMP-2, and HSP27.

In a more specific embodiment, the one or more proteins comprises fuc-PSA, SDC1 and GDF-15. In another embodiment, the one or more proteins comprises fuc-PSA, SDC1 and Tie-2. In a further embodiment, the one or more proteins comprises PSA. In yet another embodiment, the one or more proteins comprises fuc-PSA, SDC1, GDF-15, IL-6 R alpha, MMP-2 and CA15-3.

In an alternative embodiment, the one or more proteins comprise fuc-PSA and PLA2G7. In another embodiment, the one or more proteins comprises fuc-PSA, PSA, and GDF-15. In a further embodiment, the one or more proteins comprises fuc-PSA, PSA, and B7-H3. In yet another embodiment, the one or more proteins comprises fuc-PSA, PSA, GDF-15, SDC1, Tie-2 and VCAM-1. In a specific embodiment, the one or more proteins comprises fuc-PSA, PSA, GDF-15, B7-H3, Tie-2, and SDC1.

In another aspect, the measured proteins can be used further to determine certain aspects associated with prostate cancer. For example, the measured proteins can be used to identify a subject as having prostate cancer. In certain embodiments, the proteins can be used to assess prostate cancer severity (e.g., aggressive vs. non-aggressive), predict survival, and predict response to therapy.

In more particular embodiments, the one or more proteins can be used to identify patients as having aggressive prostate cancer. In certain embodiments, the measurement of total PSA, free PSA and p2PSA is used to calculate phi. The measured concentrations of the one or more proteins can be used in conjunction with phi to identify patients as having aggressive prostate cancer. In certain embodiments, % fuc-PSA can be determined and used in conjunction with the measured concentrations of the one or more proteins, as well as phi, to identify patients as having aggressive prostate cancer.

A. Measurement/Detection by Mass Spectrometry

In various embodiments the invention provides a method to identify protein biomarkers and patterns that are indicative of a disease. In various embodiments the invention provides a method to identify protein biomarkers and patterns that are indicative a disease is or may be present. In some embodiments these methods may provide objective rationale for further testing. In various embodiments the invention provides a method for the identification of a plurality of proteins from a sample, wherein each protein is correlated to one or more peptides, wherein each peptide is correlated to one or more transitions, wherein each transition comprises a Q1 mass value. In various embodiments the invention provides a method for the identification of a plurality of proteins from a sample, wherein each protein is correlated to one or more peptides, wherein each peptide is correlated to one or more transitions, wherein each transition comprises a Q1 mass value and a Q3 mass value. In various embodiments the invention provides a method for the identification of a plurality of proteins from a sample, wherein each protein is correlated to one or more peptides, wherein each peptide is correlated to one or more transitions, wherein each transition comprises a Q1/Q3 mass value pair.

As used herein, SRM stands for selected reaction monitoring. As used herein, MRM stands for multiple reaction monitoring. As used herein, PRM stands for parallel reaction monitoring. As used herein, SWATH stands for sequential window acquisition of all theoretical fragment ion spectra. As used herein, DIA stands for data-independent acquisition. As used herein, MS stands for mass spectrometry. As used herein, SIL stands for stable isotope-labeled.

As used herein, “MS data” can be raw MS data obtained from a mass spectrometer and/or processed MS data in which peptides and their fragments (e.g., transitions and MS peaks) are already identified, analyzed and/or quantified. MS data can be Selective Reaction Monitoring (SRM) data, Multiple Reaction Monitoring (MRM) data, parallel reaction monitoring (PRM) data, Shotgun CID MS data, Original DIA MS Data, MSE MS data, p2CID MS Data, PAcIFIC MS Data, AIF MS Data, XDLA MS Data, SWATH MS data, or FT-ARM MS Data, or their combinations.

In some embodiments of the present invention, based on SRM and/or MS, and/or PRM MS, allows for the detection and accurate quantification of specific peptides in complex mixtures.

Selected Reaction Monitoring or Multiple Reaction Monitoring (SRM/MRM) mass spectrometry is a technology with the potential for reliable and comprehensive quantification of substances of low abundance in complex samples. SRM is performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used including without limitation electrospray ionization, chemical ionization, electron ionization, atmospheric pressure chemical ionization, and matrix-assisted laser desorption ionization. Both the first mass analyzer and the collision cell are continuously exposed to ions from the source in a time dependent manner. Once the ions move into the third mass analyzer time dependence becomes a factor. On triple quadrupole instruments, the first quadrapole mass filter, Q1 is the primary m/z selector after the sample leaves the ionization source. Any ions with mass-to-charge ratios other than the one selected for will not be allowed to infiltrate Q1. The collision cell, denoted as “q2”, located between the first quadrapole mass filter Q1 and second quadrapole mass filter Q3, is where fragmentation of the sample occurs in the presence of an inert gas like argon, helium, or nitrogen. Upon exiting the collision cell, the fragmented ions then travel onto the second quadrapole mass filter Q3, where m/z selection can occur again. The specific pair of mass-over-charge (m/z) values associated to the precursor and fragment ions selected is referred to as a “transition”. The detector acts as a counting device for the ions matching the selected transition thereby returning an intensity distribution over time. MRM is when multiple SRM transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given analyte.

In addition to MRM, the choice of peptides can also be quantified through Parallel-Reaction Monitoring (PRM), Parallel reaction monitoring (PRM) is the application of SRM with parallel detection of all transitions in a single analysis using a high-resolution mass spectrometer. PRM provides high selectivity, high sensitivity and high-throughput to quantify selected peptide (Q1), hence quantify proteins. Again, multiple peptides can be specifically selected for each protein. PRM methodology uses the quadrupole of a mass spectrometer to isolate a target precursor ion, fragments the targeted precursor ion in the collision cell, and then detects the resulting product ions in the Orbitrap mass analyzer. Quantification is carried out after data acquisition by extracting one or more fragment ions with 5-10 ppm mass windows. PRM uses a quadrupole time-of-flight (QTOF) or hybrid quadrupole-orbitrap (QOrbitrap) mass spectrometer to carry out the peptides/proteins quantitation. Examples of QTOF include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of QOrbitrap include but are not limited to: Q Exactive™ Hybrid Quadrupole-Orbitrap Mass Spectrometer (the Thermo Scientific); or Orbitrap Fusion™ Tribrid™ (the Thermo Scientific).

Non-limiting advantages of PRM include elimination of most interferences, provides more accuracy and attomole-level limits of detection and quantification, enables the confident confirmation of the peptide identity with spectral library matching, reduces assay development time since no target transitions need to be preselected, ensures UHPLC-compatible data acquisition speeds with spectrum multiplexing and advanced signal processing.

SWATH MS is a data independent acquisition (DIA) method which aims to complement traditional mass spectrometry-based proteomics techniques such as shotgun and SRM methods. In essence, it allows a complete and permanent recording of all fragment ions of the detectable peptide precursors present in a biological sample. It thus combines the advantages of shotgun (high throughput) with those of SRM (high reproducibility and consistency).

In some embodiments, the developed methods herein can be applied to the quantification of polypeptides(s) or protein(s) in biological sample(s), such as urine and/or serum. Any kind of biological samples comprising polypeptides or proteins can be the starting point and be analyzed by the methods herein. Indeed, any protein/peptide containing sample can be used for and analyzed by the methods produced here (e.g., tissues, cells). The methods herein can also be used with peptide mixtures obtained by digestion. Digestion of a polypeptide or protein includes any-kind of cleavage strategies such as enzymatic, chemical, physical or combinations thereof.

In some embodiments, the analysis and/or comparison is performed on protein samples of wild-type or physiological/healthy origin against protein samples of mutant or pathological origin.

B. Measurement/Detection by Immunoassays

In specific embodiments, the proteins of the present invention can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. The biospecific capture reagents can, in some embodiments, bind all forms of the biomarker, e.g., PSA and its post-translationally modified forms (e.g., glycosylated form). In other embodiments, the biospecific capture reagents bind the specific biomarker and not similar forms thereof.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the protein biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarker; and detecting binding of the antibodies, or antigen binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

For example, the level of a biomarker in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the protein. The detection can be performed using a second antibody to bind to the capture antibody complexed with its target biomarker. A target biomarker can be an entire protein, or a variant or modified form thereof. Kits for the detection of proteins as described herein can include pre-coated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

The present disclosure also provides methods for detecting protein in a sample obtained from a subject, wherein the levels of expression of the proteins in a biological sample are determined simultaneously. For example, in one embodiment, methods are provided that comprise: (a) contacting a biological sample obtained from the subject with a plurality of binding agents that each selectively bind to one or more biomarker proteins for a period of time sufficient to form binding agent-biomarker complexes; and (b) detecting binding of the binding agents to the one or more biomarker proteins. In further embodiments, detection thereby determines the levels of expression of the biomarkers in the biological sample; and the method can further comprise (c) comparing the levels of expression of the one or more biomarker proteins in the biological sample with predetermined threshold values, wherein levels of expression of at least one of the biomarker proteins above or below the predetermined threshold values indicates, for example, the subject has prostate cancer, the severity of prostate cancer, and/or is/will be responsive to prostate cancer therapy. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above-described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, lectins, peptides, aptamer, etc., combinations thereof) to form a biomarker:capture agent complex. The complexes can then be detected and/or quantified. A subject can then be identified as having aggressive prostate cancer based on a comparison of the detected/quantified/measured levels of biomarkers to one or more reference controls as described herein.

In one method, a first, or capture, binding agent, such as an antibody that specifically binds the protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In a more specific embodiment, a biotinylated lectin that specifically binds a biomarker can be added to a patient sample and a streptavidin labeled fluorescent marker that binds the biotinylated lectin bound to the biomarker is then added, and the biomarker is detected.

In another embodiment, the assay is a competitive binding assay, wherein labeled protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, predetermined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

In several embodiments, the biomarkers of the present invention may be detected by means of an electrochemiluminescent assay developed by Meso Scale Discovery (Gaithersburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at −620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

C. Measurement/Detection By Other Detection Methods

The proteins of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In particular embodiments, the protein biomarker proteins of the present invention can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of blood serum or plasma are dropped on the chip array. Protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

### III. Treatment Methods

In another aspect, the present invention provides a prostate cancer therapy or therapeutic interventions practically applied following the measurement/detection of biomarker glycopeptides. In particular embodiments, therapeutic intervention comprises prostatectomy, radiation therapy, cryotherapy (also referred to as cryosurgery or cryoablation), hormone therapy, chemotherapy, immunotherapy and combinations thereof.

Prostatectomy includes radical prostatectomy (open (radical retropubic prostatectomy or radical perineal prostatectomy) or lateral (laparoscopic radical prostatectomy including robotic-assisted), and transurethral resection of the prostate (TURP).

Radiation therapy includes external beam radiation (three-dimensional conformal radiation therapy (3D-CRT), intensity modulated radiation therapy (IMRT), stereotactic body radiation therapy (SBRT), proton beam radiation therapy) and brachytherapy (internal radiation) (permanent (low dose rate or LDR) brachytherapy or temporary (high dose rate or HDR) brachytherapy).

Hormone therapy (androgen suppression therapy) includes orchiectomy (surgical castration), luteinizing hormone-release hormone (LHRH) agonists (e.g., leuprolide, goserelin, triptorelin, histrelin), LHRH antagonists (e.g., degareli), treatment to lower androgen levels from the adrenal glands (e.g., abiraterone, ketoconazole), anti-androgens (e.g., flutamide, bicalutamide, nilutamide, enzalutamide, apalutamide), and estrogens.

Chemotherapy includes treatment with compounds including, but not limited to, docetaxel, cabazitaxel, mitoxantrone, and estramustine.

Immunotherapy includes, but is not limited to, a cancer vaccine (e.g., sipuleucel-T), as well as immune checkpoint inhibitors (e.g., PD-1 inhibitors including pembrolizumab). Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB00010718C (anti-PDL1 antibody) and Yervoy/ipilimumab (anti-CTLA-4 checkpoint inhibitor).

A prostate therapeutic intervention can comprise a targeted therapy including poly(ADP)-ribose polymerase (PARP) inhibitor (e.g., niraparib (zejula), olaparib (lynparza), and rucaparib (rubraca)).

Other therapeutic interventions for prostate cancer include an androgen receptor (AR)-targeted therapy (e.g., enzalutamide, ARN-509, ODM-201, EPI-001, hydrazinobenzoylcurcumin (HBC), aberaterone, geleterone, and seviteronel), an antimicrotubule agent, an alkylating agent and an anthracenedione.

In particular embodiments, a therapeutic intervention for prostate cancer can include the administration of drugs including, but not limited to, Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate), Zytiga (Abiraterone Acetate).

### IV. Kits

In another aspect, the present invention provides kits for detecting one or more biomarker proteins. The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of one or more proteins including by any one or more of mass spectrometry, antibody method, antibodies, lectins, nucleic acid aptamer method, nucleic acid aptamers, immunoassay, ELISA, immunoprecipitation, SISCAPA, Western blot, or combinations thereof; and (b) reagents and instructions for sample processing, preparation and biomarker protein measurement/detection. The kit can further comprise (c) instructions for using the kit to measure biomarker proteins in a sample obtained from the subject.

In particular embodiments, the kit comprises reagents necessary for processing of samples and performance of an immunoassay. In a specific embodiment, the immunoassay is an ELISA. Thus, in certain embodiments, the kit comprises a substrate for performing the assay (e.g., a 96-well polystyrene plate). The substrate can be coated with antibodies specific for a biomarker protein. In a further embodiment, the kit can comprise a detection antibody including, for example, a polyclonal antibody specific for a biomarker protein conjugated to a detectable moiety or label (e.g., horseradish peroxidase). The kit can also comprise a standard, e.g., a human protein standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having protein biomarker capture reagents attached thereon. The kit may further comprise a means for detecting the protein biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In a specific embodiment, a kit comprises one or more of (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker.

In certain embodiments, a subject can be diagnosed by adding a biological sample (e.g., serum) from the patient to the kit and detecting the relevant protein biomarkers conjugated with antibodies and/or lectins, specifically, by a method which comprises the steps of: (i) collecting serum from the patient; (ii) adding serum from patient to a diagnostic kit; and, (iii) detecting the protein biomarkers conjugated with antibodies/lectins. If the biomarkers are present in the sample, the antibodies/lectins will bind to the sample, or a portion thereof. In other kit and diagnostic embodiments, serum will not be collected from the patient (i.e., it is already collected). Serum or other samples can be collected from subject of varying ages. Indeed, in other embodiments, the sample may comprise a urine, blood, plasma sweat, tissue, blood or a clinical sample.

The kit can also comprise a washing solution or instructions for making a washing solution, in which the combination of the capture reagents and the washing solution allows capture of the protein biomarkers on the solid support for subsequent detection by, e.g., antibodies/lectins or mass spectrometry. In a further embodiment, a kit can comprise instructions for suitable operational parameters in the form of a label or separate insert. For example, the instructions may inform a consumer about how to collect the sample, etc. In yet another embodiment, the kit can comprise one or more containers with protein biomarker samples, to be used as standard(s) for calibration or normalization. Detection of the markers described herein may be accomplished using a lateral flow assay.

In certain embodiments, the kit comprises reagents and components necessary for performing an electrochemiluminescent ELISA.

In another aspect the present invention provides kit. In particular embodiments, a kit comprises (a) monoclonal antibodies that each specifically bind a biomarker protein of interest; (b) biotinylated immunoglobulin G detection antibodies; (c) biotinylated lectins that specifically bind glycosylated forms of a biomarker protein of interest (e.g., PSA); and (d) streptavidin labeled fluorescent markers. In a further embodiment, the kit further comprises (e) magnetic beads for conjugating to monoclonal antibodies that each specifically bind a biomarker protein of interest. In a specific embodiment, the biomarker protein of interest comprises one or more of PSA, fuc-PSA, p2PSA, B7-H3, PLA2G7, GDF-15, IL-6 R alpha, SDC1, VCAM-1, sTie-2, IL-16, CA15-3, MMP-2, and HSP27.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Background: Current PSA-based tests used to detect prostate cancer (PCa) lack sufficient specificity, leading to significant overdetection and overtreatment. Our previous studies showed that serum fucosylated PSA (Fuc-PSA) and soluble TEK receptor tyrosine kinase (Tie-2) had the ability to predict aggressive (AG) PCa. Additional biomarkers are needed to address this significant clinical problem.

Methods: A comprehensive Pubmed search followed by multiplex immunoassays identified candidate biomarkers associated with AG PCa. Subsequently, multiplex and lectin-based immunoassays were applied to a case-control set of sera from subjects with AG PCa, low risk PCa, and non-PCa (biopsy negative). These candidate biomarkers were further evaluated for their ability as panels to complement the prostate health index (phi) in detecting AG PCa.

Results: When combined through logistic regression, two panel of biomarkers achieved the best performance: 1) phi, Fuc-PSA, SDC1, and GDF-15 for the detection of AG from low risk PCa and 2) phi, Fuc-PSA, SDC1, and Tie-2 for the detection of AG from low risk PCa and non-PCa, with noticeable improvements in ROC analysis over phi alone (AUCs: 0.942 vs 0.872, and 0.934 vs 0.898, respectively). At a fixed sensitivity of 95%, the panels improved specificity with statistical significance in detecting AG from low risk PCa (76.0% vs 56%, p=0.029), and from low risk PCa and non-PCa (78.2% vs 65.5%, p=0.010).

Conclusions: Multivariate panels of serum biomarkers identified in this study demonstrated clinically meaningful improvement over the performance of phi, and warrant further clinical validation, which may contribute to the management of PCa.

### Introduction

The goal of this study was to identify and combine serum proteomic biomarkers into a panel for distinguishing AG PCa from low-risk cancer. Two biomarkers, fucosylated PSA (Fuc-PSA) and soluble TEK receptor tyrosine kinase (Tie-2), were discovered in our previous Early Detection Research Network (EDRN) studies with demonstrated ability to predict AG PCa [6-8]. Fucosylated proteins have been found to be associated with cancer and potentially used as tumor markers [9-11]. An example is the fucosylated alpha-fetoprotein (AFP-L3), an FDA cleared diagnostic test for assessing the risk of developing hepatocellular carcinoma [12, 13]. We developed quantitative lectin-based immunoassays for serum Fuc-PSA and demonstrated that Fuc-PSA could be an effective biomarker to detect AG PCa [7, 8]. Serum angiogenic factors are potential candidates for prognostic biomarkers in PCa [14, 15]. Tie-2 is a transmembrane tyrosine kinase receptor for angiopoietins and is crucial for angiogenesis and vascular maintenance [16, 17]. We previously demonstrated that serum levels of Tie-2 were elevated in PCa patients with GS 8-10 [6]. In this study, we evaluated whether combinations of Fuc-PSA, Tie-2, and/or other selected biomarkers from an expanded list of candidates combined with current FDA approved PSA-based test modalities, specifically, prostate health index (phi) [18], could improve their diagnostic ability for the detection of AG PCa.

Materials and Methods

Study Design. We performed a comprehensive literature search and identified 22 additional candidate biomarkers reported to be associated with AG PCa. We tested these 22 candidates with multiplex immunoassays in a well-characterized in-house clinical sample set. Based on bootstrap area-under-curve (AUC) analysis, the list was reduced to the 10 best performing biomarkers with respect to the combined criteria of a relatively high AUC mean and a relatively low AUC standard deviation (STD) in separating low risk versus AG PCa. In this study, using a case-control sample set, we evaluated whether these 10 biomarkers as well as Tie-2 and Fuc-PSA in combined use with current FDA approved PSA-based test modalities, specifically, prostate health index (phi) [18], could further improve the detection of AG PCa.

Specimens. Specimens for this study were collected at Beth Israel Deaconess Hospital, Harvard Medical School from 2005 to 2008 as part of the prospective EDRN Clinical Validation Center cohort [19]. Eligibility for the EDRN cohort included patient age greater than 40 years, no prior prostate surgery, biopsy or history of PCa, availability of serum samples with corresponding clinical data, and completion of biopsy under transrectal ultrasound guidance using a standard template after enrolment. Serum samples were collected prior to initial biopsy and stored at −80° C. until analysis. Serum samples obtained from 90 patients, including 60 patients with histologically diagnosed PCa and 30 biopsy negative controls were included in this study with institutional approval. For the current study, GS was used as a surrogate for PCa aggressiveness. Consistent with the majority view in the literature [4, 5, 20], a tumor with a GS 7 or greater was considered as AG PCa and GS 6 or less as low risk PCa.

Reagents. Human Magnetic Luminex Assays (LXSAHM-15, LXSAHM-08, and LXSAHM-02) were purchased from R&D Systems (Minneapolis, MN). Magnetic COOH beads, amine coupling kits, and Bio-Plex Pro Reagent kits were purchased from Bio-Rad Laboratories (Hercules, CA). NHS and Sulfo-NHS, EDC, EZ-Link™ Sulfo-NHS-Biotin, and Zeba™ Spin Desalting Columns were purchased from Thermo Scientific (Rockford, IL). Agarose bound Aleuria Aurantia Lectin (AAL) was purchased from Vector Laboratories (Burlingame, CA). Pierce™ BCA Protein Assay Kit was purchased from Thermo Fisher Scientific (Waltham, MA).

Multiplex immunoassays. Human Magnetic Luminex Assays were performed following the manufacturer's protocols on the Bio-Plex 200 system. Samples were diluted 1:2 (the initial 15-plex and the finalized 8-plex assays) or 1:50 (2-plex assay) in the calibrator diluent. Calibration curves were established using 7 calibrators in a 3-fold dilution series in the calibrator diluent derived from a mixture of the highest standard points of multiple recombinant proteins. The highest standards were 215.8, 883.2, 4.8, 25.6, 63.4, 1977.9, 169.7, and 10.3 ng/mL for CD276 molecule (B7-H3), phospholipase A2 group VII (PLA2G7), growth differentiation factor 15 (GDF-15), interleukin-6 receptor subunit alpha (IL-6 R alpha), Syndecan-1 (SDC1), vascular cell adhesion molecule 1 (VCAM-1), TEK receptor tyrosine kinase (Tie-2), and interleukin 16 (IL-16), respectively (8-plex); 40 U/mL and 57.2 ng/mL for cancer antigen (CA 15-3) and matrix metallopeptidase (MMP-2), respectively (2-plex). Heat shock 27 kDa protein (HSP27) assay (1-plex) was carried out with the sample diluted 1:4 in the standard diluent, and the calibration curve was established using 7 calibrators in 2.5-fold dilution series in the standard diluent. The highest standard of the recombinant protein in the assay was 3.0 ng/mL. Immunoassays were performed in duplicate on 96-well Bio-Plex flat bottom plates. All samples were randomized with respect to their plate locations.

Calibration curves were constructed with Bio-Plex Manager Software version 6.1.1 using a 5-parametric (5-PL) nonlinear logistic regression curve fitting model. Assay sensitivity (limit of blank, LOB) was defined as the concentration of analyte corresponding to the median fluorescent intensity (MFI) of the background plus two STDs of the mean background MFI. Intra-assay precision was calculated as the coefficient of variance (% CV) on 4 replicates of pooled normal sera (S7023 from Sigma-Aldrich) on a single assay plate. Inter-assay precision was calculated as the % CV from 3 replicates. The assay working dynamic range was defined as the range between the lower limit of quantification (LLOQ) and the upper limit of quantification (ULOQ) in which an assay is both precise (intra-assay % CV≤10% and inter-assay % CV≤15%) and accurate (80-120% recovery).

Fucosylated PSA. Lectin-based immunoassays for Fuc-PSA to detect AG PCa were developed and described previously [8]. In this study, we used agarose bound AAL beads to enrich Fucosylated proteins from patient sera then tested PSA with the Hybritech PSA assay on the Access 2 Immunoassay Analyzer (Beckman Coulter, Inc.) [8, 18].

PSA and phi analysis. Serum samples were analyzed for total PSA, free PSA (fPSA), and [−2]proPSA (p2PSA) [18, 21] on the Access 2 Immunoassay Analyzer (Beckman Coulter, Inc). Prostate health index (phi) was calculated with the equation, (p2PSA/fPSA)×PSA^{1/2}.

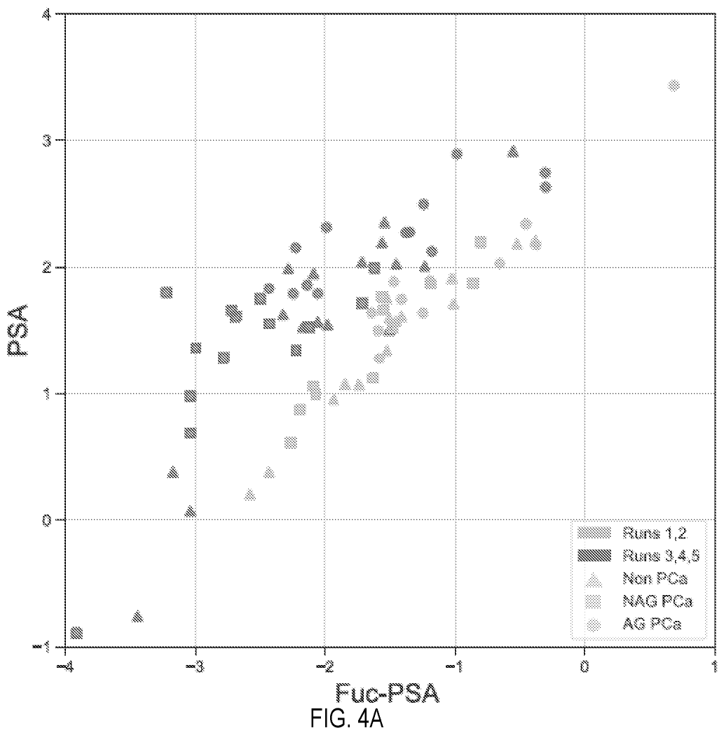

Statistical Analysis. Biomarker data were transformed prior to analysis (log-transformation followed by z-score). To correct for an observed batch-effect in Fuc-PSA measurement, z-scores of log-transformed Fuc-PSA data were computed separately for each of the two batches before being merged together. Scatterplots of the Fuc-PSA values before and after correction against total PSA, which was not affected by the batches, confirmed negligible residual differences (FIG. 4). Furthermore, as shown in the same plots, with block-randomization of samples, the distribution of samples between the batches did not confound the sample clinical phenotype.

Diagnostic performance of individual biomarkers to differentiate AG from low risk PCa, and AG from low risk PCa and non-PCa were evaluated first by univariate analysis based on estimated AUCs from receiver-operating characteristic (ROC) curve analysis. To evaluate the statistical stability of results, bootstrap resampling (n=1,000) [19, 22, 23] was used to estimate the mean and STD of AUCs of individual biomarkers.

Multivariate analyses were further carried out to evaluate the complementary values of biomarkers to established clinical test modalities with respect to the detection of AG PCa. With the limited number of available samples, we chose to evaluate only linear combinations using logistic regression of up to three novel markers with the clinical test phi and to identify panels of biomarkers with the greatest improvement in ROC/AUC over that of phi alone. This was done for both the detection of AG from low risk PCa, and from low risk PCa and non-PCa. In addition, we also specifically evaluated the value of Fuc-PSA in complementing phi as a two-marker panel. Bootstrap resampling was used to estimate 95% confidence intervals of ROC/AUCs.

Considering the potential clinical utility of a test to separate AG from low risk PCa (and/or non-PCa,), a very high sensitivity will likely be required to achieve a clinically acceptable negative predictive value for patient safety. For the identified multivariate panels, we therefore further assessed improvement in specificity at a fixed high level of sensitivity. Differences between groups were assessed using the Mann-Whitney U test. Statistical significance was considered at p<0.05. Statistica 13 (StatSoft), GraphPad Prism 6 (GraphPad Software), MedCalc (MedCalc Software, Ostend, Belgium), and inhouse-developed Python scripts using library functions from matplotlib (2.2.3), NumPy (1.16.5), pandas (0.24.2), seabom (0.9.0), scikit-learn (1.16.5) and SciPy (1.2.1) were used for statistical analyses. Other than specifically indicated, confidence intervals (CI) of AUCs and other performance measurement were based on bootstrap estimation.

Results

Patient characteristics. A total of 90 patients including 60 PCa cases and 30 non-PCa controls were included in this study. Among the PCa cases, 30 were biopsy GS≤6 and the other 30 were GS≥7. The non-PCa patients were biopsy negative controls. Among all the samples, one case was excluded due to a specimen quality issue. Of the remaining 89 samples, 7 had no PSA-related assay data and 2 had no Fuc-PSA data due to insufficient quantity for measurement. Consequently, other than the tabulated descriptive statistics and scatterplots of the individual biomarkers with the 89 samples, all statistical analyses were performed using 80 samples (25 AG and 25 low risk PCa, and 30 non-PCa) that had no missing data across all biomarkers.

Following an extended-pattern prostate biopsy schema [19], 98.8% of 80 patients underwent 12-core or greater biopsy with a median (range) number of 12 (8 to 20). Among 19 cases that went on to prostatectomy and had available pathologic GS, there were 3 cases with GS 6 upgraded to GS 7, and 3 with GS 8 and 1 with GS 9 downgraded to GS 7 on prostatectomy pathology. Detailed clinicopathologic characteristics of the study cohort, including diagnosis, age, race, family history of PCa, DRE (digital rectal examination), GS, clinical stage, PSA, % fPSA, and phi are shown in Table 1.

Identification of biomarkers for multiplex immunoassay. In addition to evaluating two previously identified serum biomarkers (Fuc-PSA and Tie-2) [6-8], additional serum biomarkers with potential relevance to AG PCa were curated through a comprehensive literature search in PubMed. The inclusion of these biomarkers in our multiplex imunoassay panels took into consideration the reported clinically relevant performance characteristics and strength of evidence, biological feasibility supported by existing knowledge/databases such as results from large-scale genomic and proteomics analysis, ability to complement other biomarkers in the selection, their relative abundance in human serum samples, and the likelihood of available resources and constraints (antibodies, concentration in target specimens, etc.). Through in silico analysis, a total of 22 candidate biomarkers were selected to be assessed using a Bio-Plex 200 suspension array system (Bio-Rad) as described previously [24, 25] in 40 sera from patients diagnosed with AG or low risk PCa and benign prostate diseases, which were collected from JHH with institutional approval (data not shown). Ten candidate biomarkers (B7-H3, PLA2G7, GDF-15, IL-6 R alpha, SDC1, VCAM-1, IL-16, CA15-3, MMP-2 and HSP27) and one previously reported biomarker (Tie-2) were further evaluated using multiplex immunoassays in the 90 patient sera collected from Beth Israel Deaconess Hospital. The multiplex immunoassays had acceptable analytical performance with recoveries of 98% to 104%, intra-assay precision of 0.8% to 4.8%, inter-assay precision of 0.8% to 4.2%, wide dynamic concentration ranges (>2 logs) defined by LLOQ and ULOQ, and low LOBs for target protein quantification (data not shown).

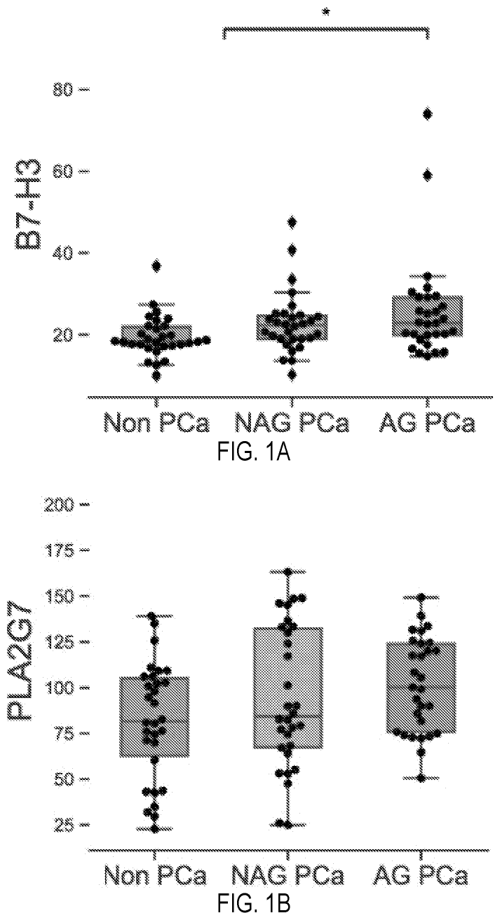

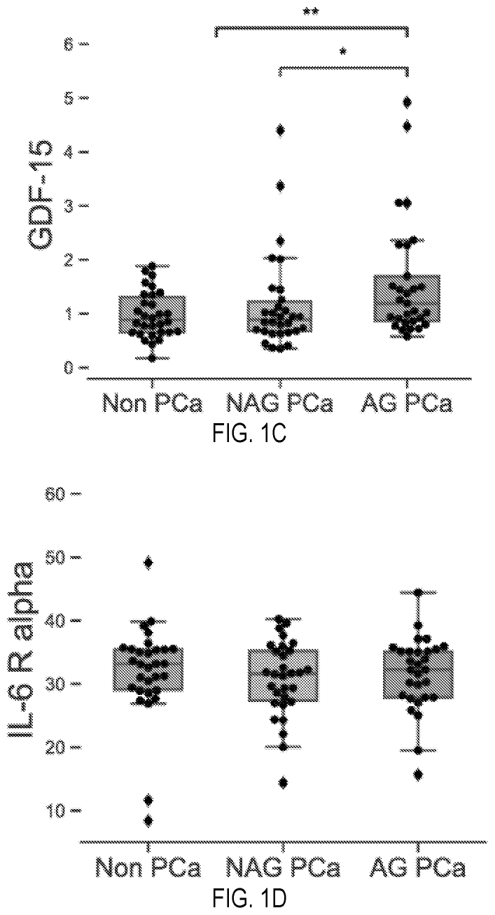

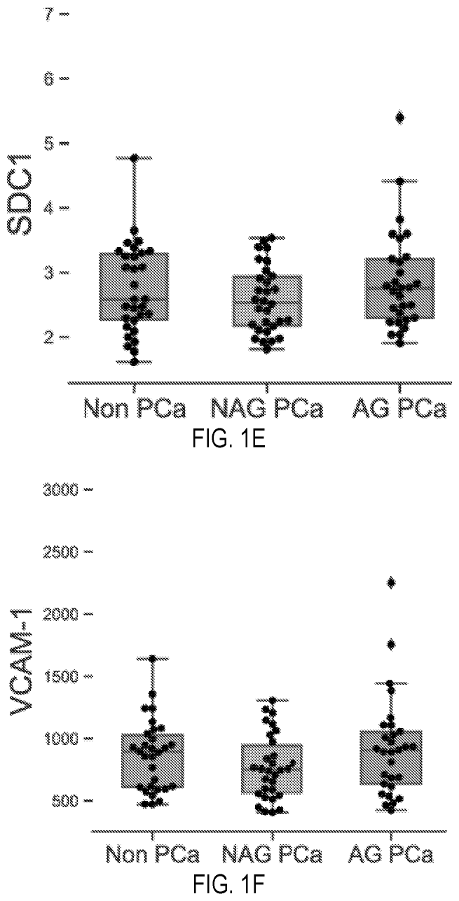

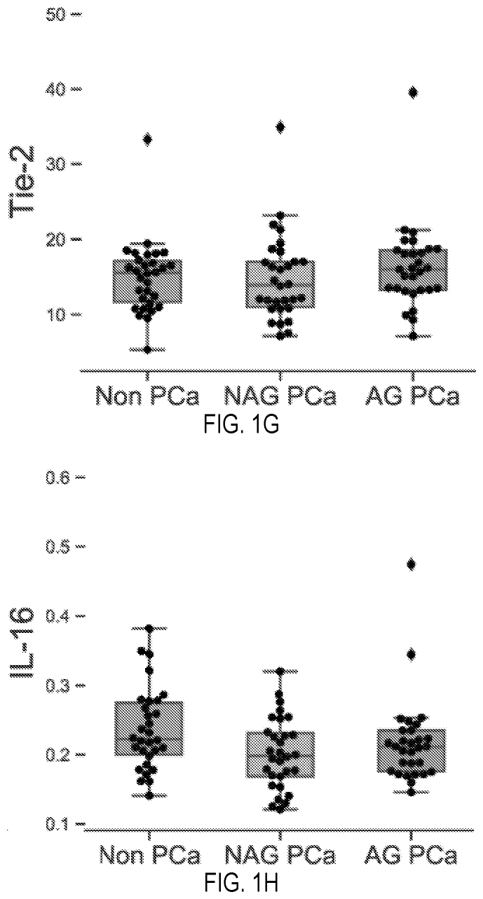

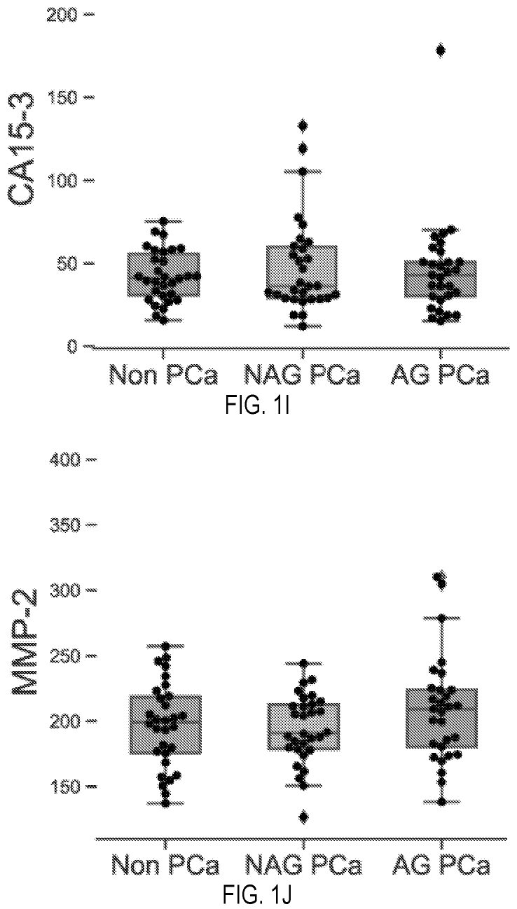

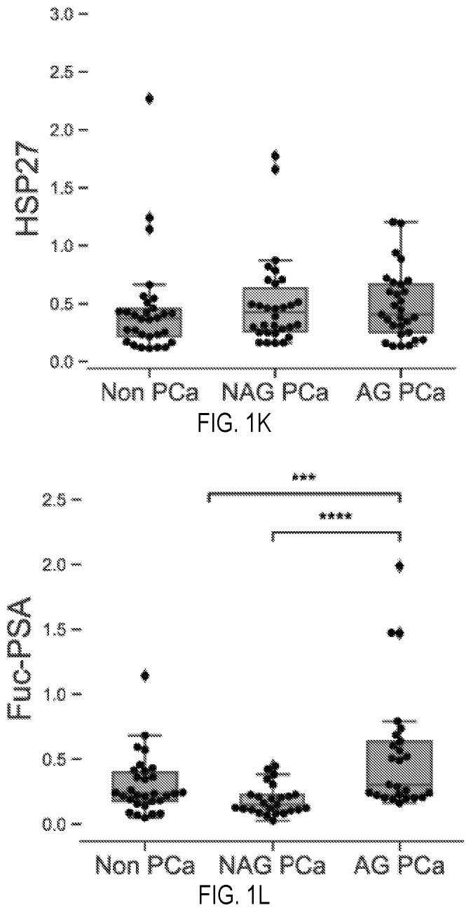

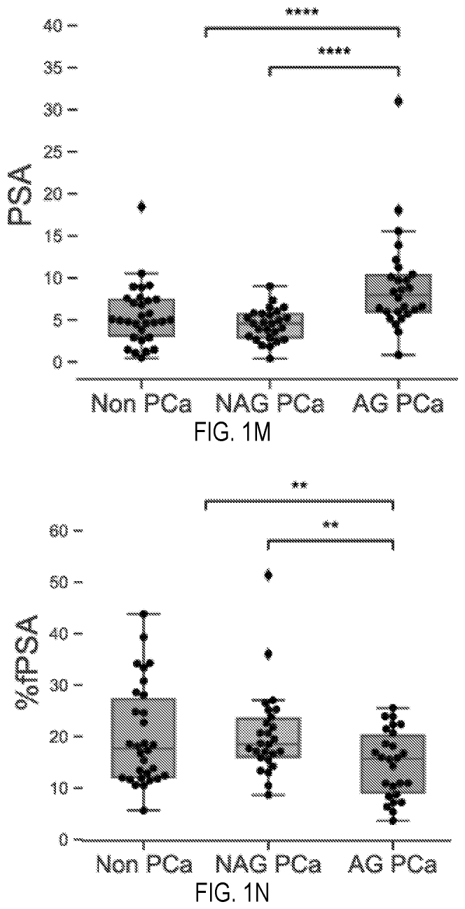

Univariate evaluation of biomarker selection. Serum concentrations of individual biomarkers were compared among AG and low risk PCa patients as well as non-PCa controls (FIG. 1A-1O and Table 3). Biomarkers that individually showed a statistically significant difference in serum levels between AG and low risk PCa patients included GDF-15 (p<0.01), % fPSA (p<0.05, lower in AG), and Fuc-PSA, PSA, and phi (all at p<0.0001). When comparing AG PCa to the combined group of low risk PCa and non-PCa, biomarkers with significant differences included B7-H3 (p<0.05), % fPSA (p<0.05, lower in AG), GDF-15 (p<0.01), Fuc-PSA (p<0.001), and PSA and phi (both at p<0.0001).

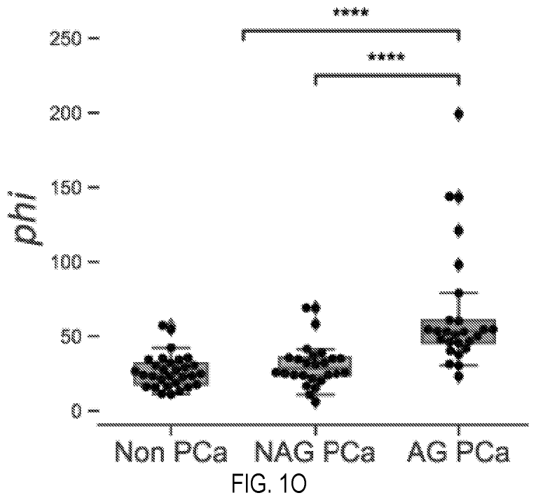

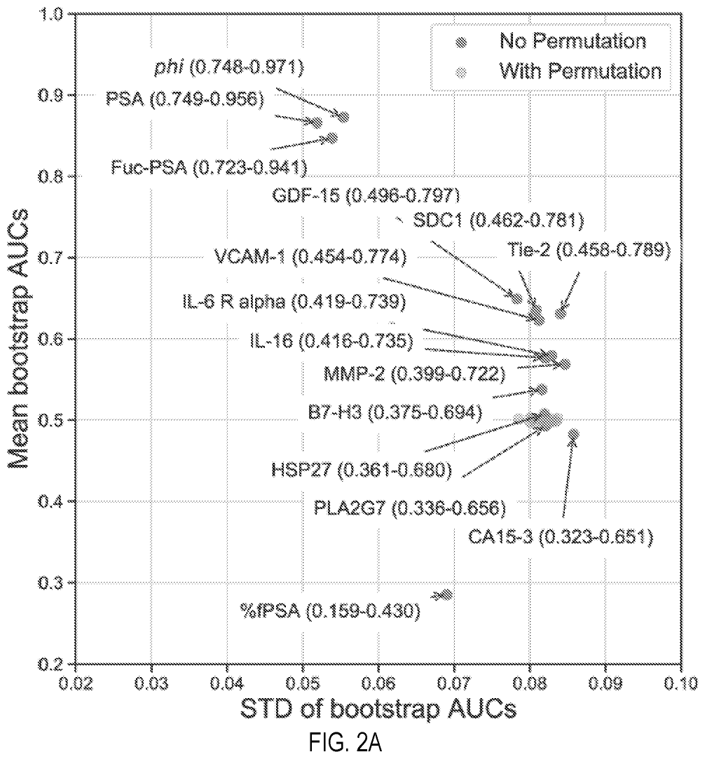

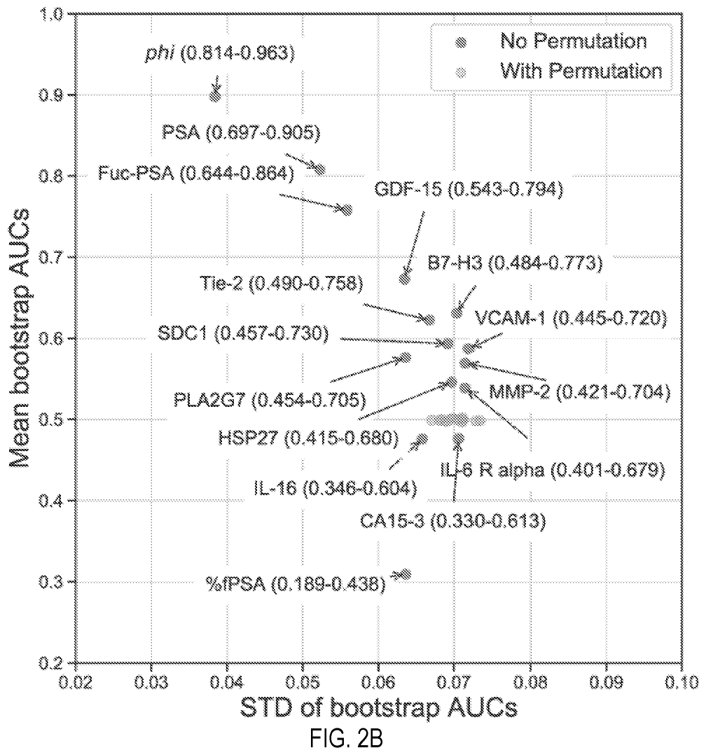

To provide a more clinically relevant comparison, the AUCs from ROC analysis were also estimated. The best biomarkers to separate AG from low risk PCa were phi (AUC=0.872), PSA (0.866), Fuc-PSA (0.848), % fPSA (0.714]), GDF-15 (0.651), SDC1 (0.637), Tie-2 (0.635), and VCAM-1 (0.626), and to separate AG from low risk PCa and non-PCa were phi (AUC=0.898), PSA (0.807), Fuc-PSA (0.757), % fPSA (0.691), GDF-15 (0.673), B7-H3 (0.630), Tie-2 (0.620), and SDC1 (0.593). To further evaluate the statistical stability of biomarker performance within this sample set, FIGS. 2A and 2B show the bootstrap estimated mean and STDs for the AUCs of individual biomarkers. PSA related assays, including phi, had the best and most stable diagnostic performance in this specific cohort of patient samples.

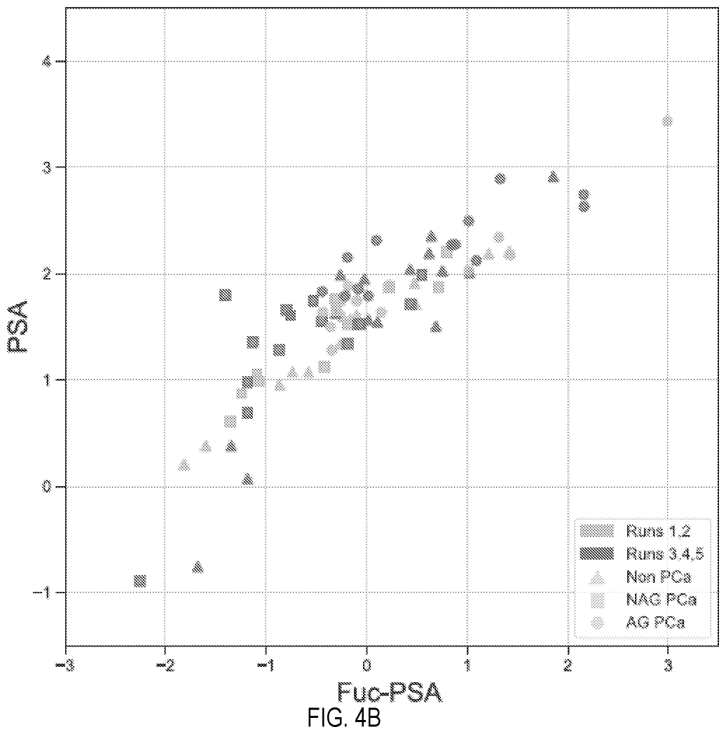

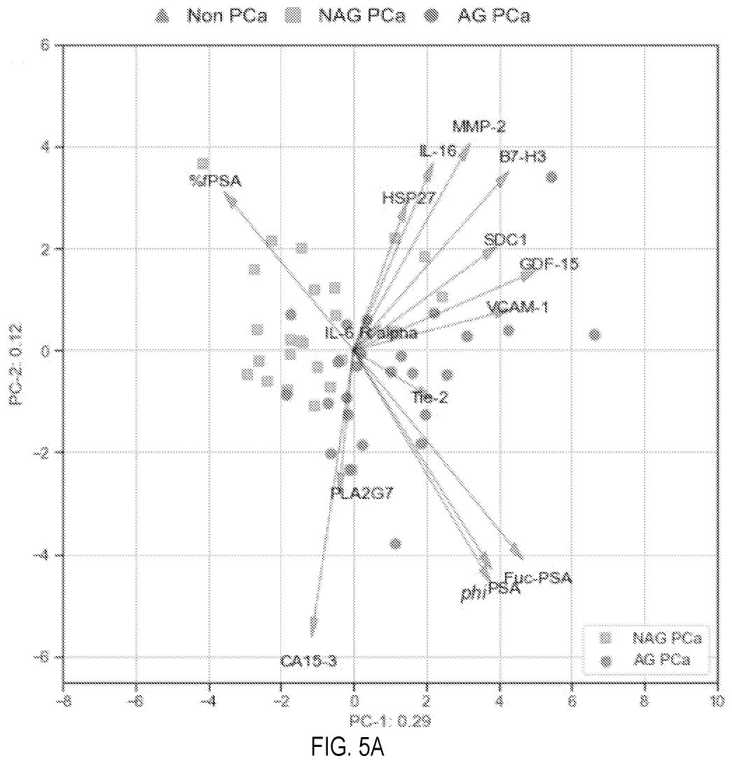

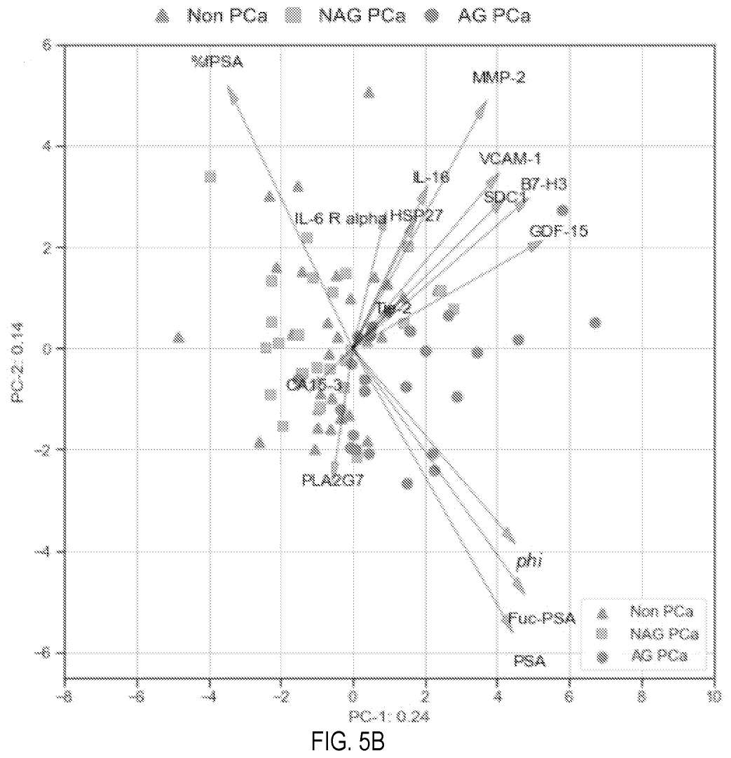

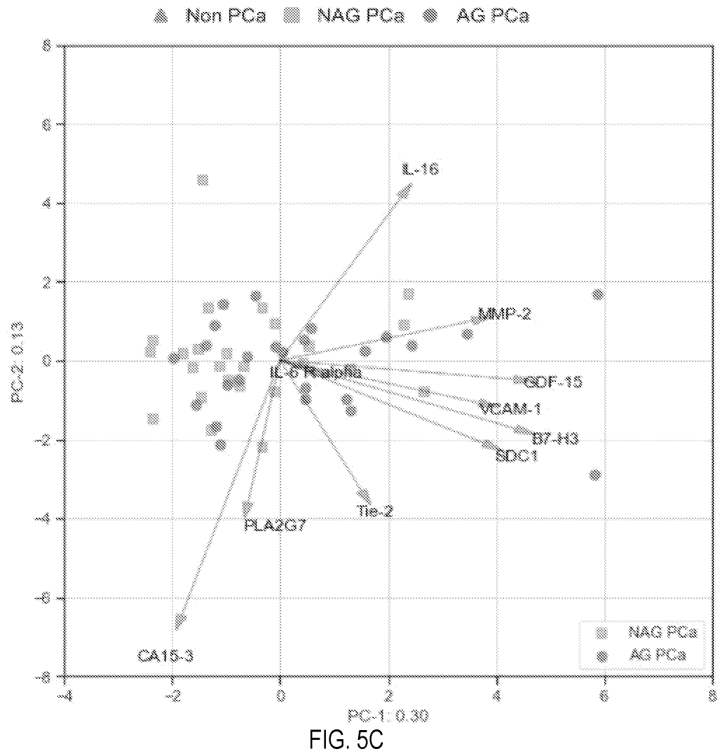

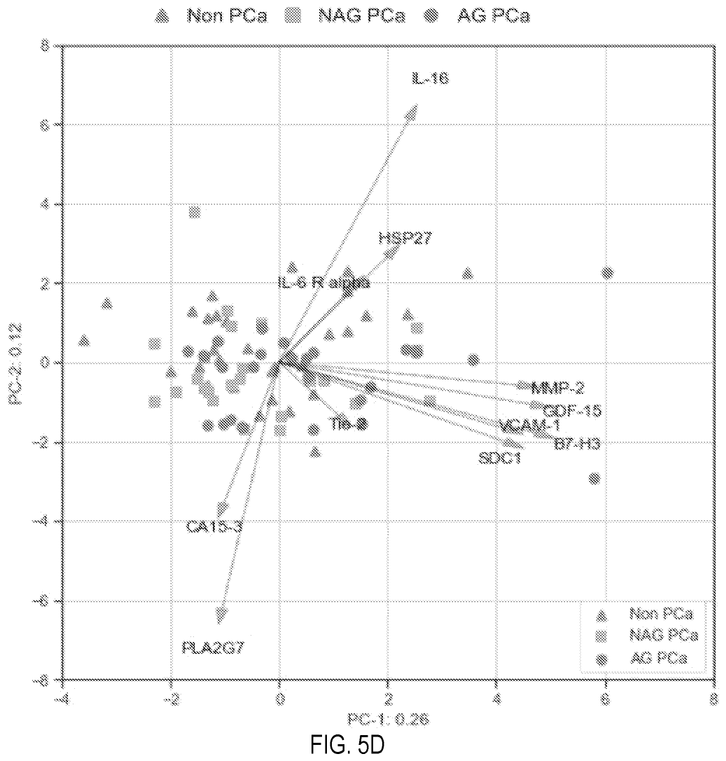

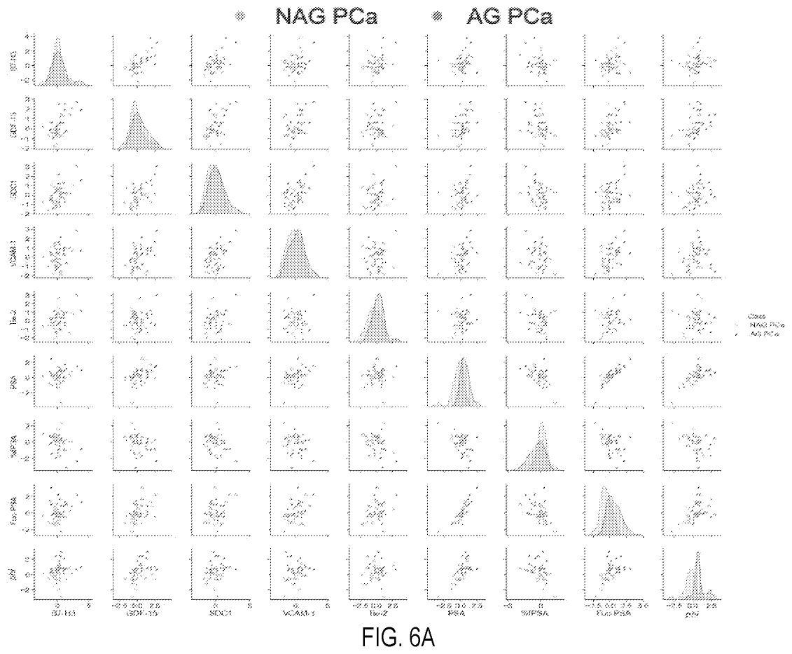

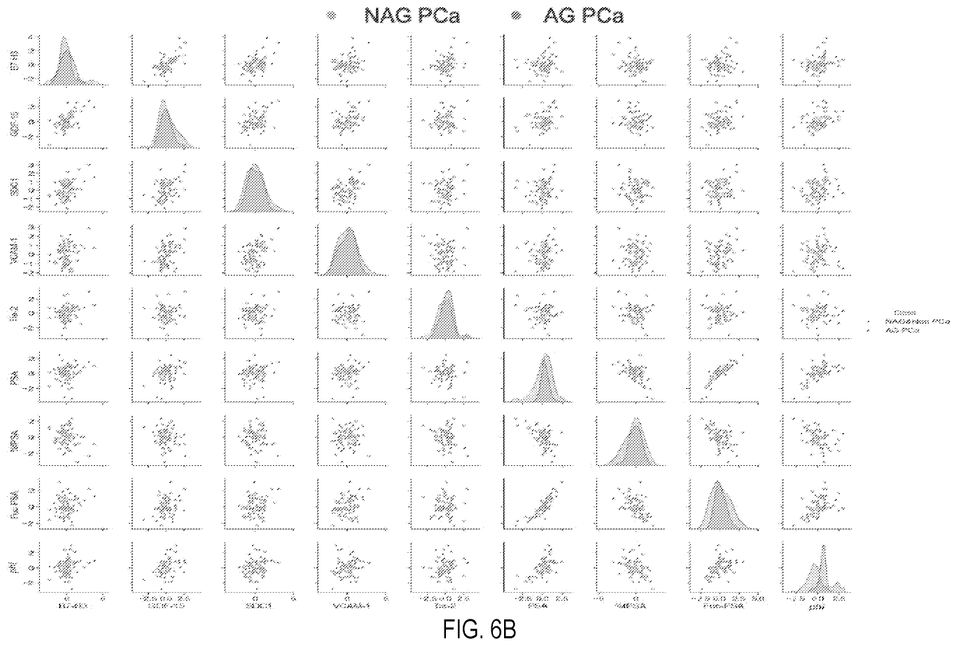

Multivariate evaluation of biomarker complementarity. In order to depict the strengths and relative relationships among the multiple biomarkers with respect to their ability to separate AG from either low risk PCa only or from low risk PCa and non-PCa, the biomarker data were used unsupervised through principal component analysis (PCA) to generate biplots [26] (FIG. 5) in which the contributions (loadings) of individual biomarkers to the first and second principal components (PCs) were represented as vectors superimposed on the PCA plot of individual patient samples. As expected, when PSA, % fPSA, Fuc-PSA, and phi were included with the other candidate biomarkers in PCA analysis, the AG samples were reasonably well separated from either low risk PCa only (FIG. 5A) or from low risk PCa and non-PCa (FIG. 5B). Interestingly, for this particular sample set, there was no obvious separation between the low risk PCa and non-PCa samples. In the bioplots, the loading vectors of several non-PSA related candidate biomarkers, such as Tie-2, GDF-15, SDC1, B7-H7, VCAM-1 as a cluster, were at angles to those of the PSA-based biomarkers yet still pointed to the direction that would complement the PSA-based biomarkers in separating AG PCa and low risk PCa or non-PCa samples, indicating potential complementary value to the PSA-related tests. When a similar analysis was performed without the PSA-related assays, the clinical groups overlapped significantly (FIGS. 5C-5D), with B7-H3, SDC1, GDF-15, Tie-2, and VCAM-1 retaining some level of contribution towards the separation of AG PCa samples. Tabulated pair-wise scatterplots (FIG. 6A-6B) of these biomarkers and the PSA-related biomarkers (nine in total) offer visualization of potential pair-wise complementary relations or the lack thereof among them.

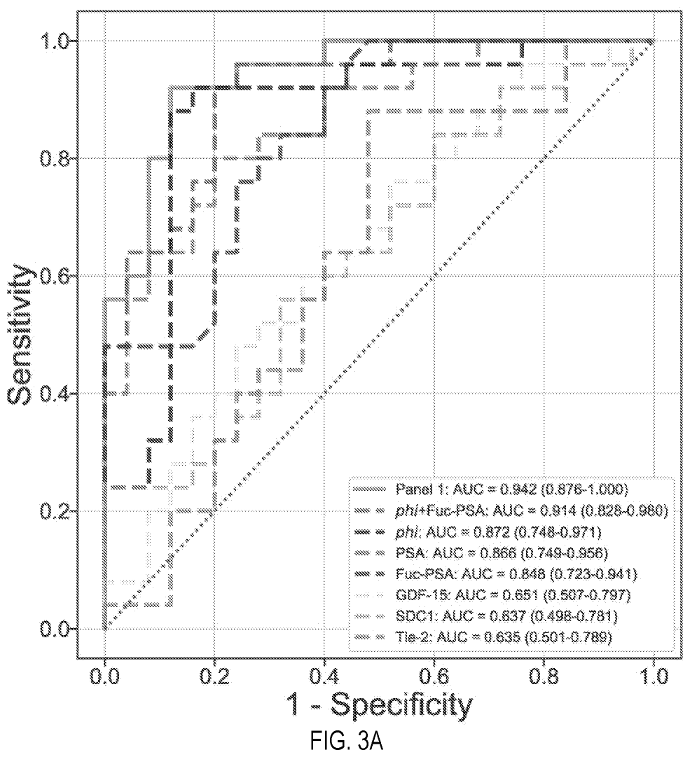

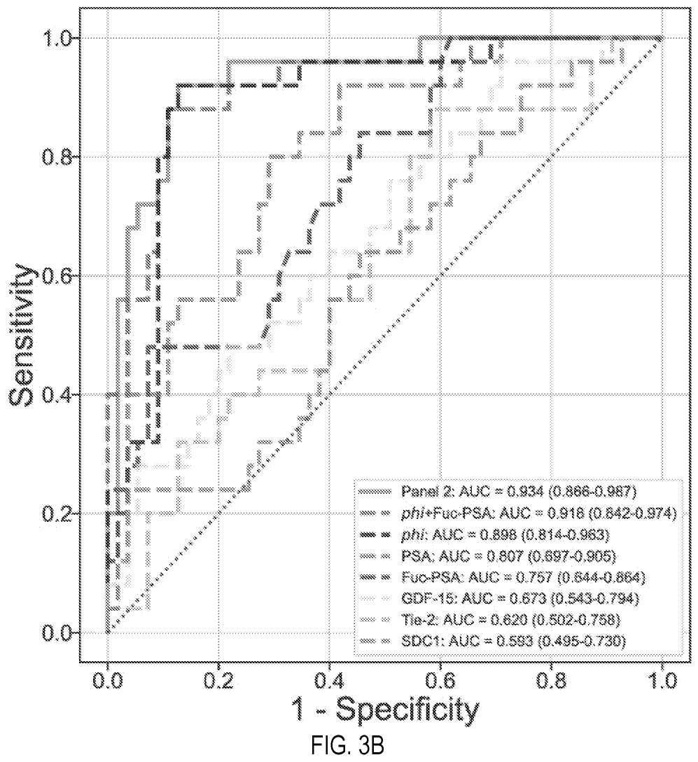

Using logistic regression, two panels were identified among all panels of up to 4 markers (including phi but excluding PSA and % fPSA) to offer the most improvement in ROC/AUC over that of phi alone in separating AG from low risk PCa (AUC_{phi+Fuc-PSA+SDC1+GDF-15}=0.942 vs AUCₚₕᵢ=0.872) or from low risk PCa and non-PCa (AUC_{phi+Fuc-PSA+SDC1+Tie-2}=0.934 vs AUCₚₕᵢ=0.898) (FIGS. 3A and 3B). In addition, the combination of phi and Fuc-PSA also improved the performance of phi in separating AG from low risk PCa (AUC_{phi+Fuc-PSA}=0.914) or from low risk and non-PCa (AUC_{phi+Fuc-PSA}=0.918). The improvement from these panels over phi were statistically significant comparing the means of bootstrap estimated AUCs (AUC_{phi+Fuc-PSA+sDC1+GDF-15}=0.945 or AUC_{phi+Fuc-PSA}=0.916 vs AUCₚₕᵢ=0.873, both p<0.0001 for AG vs low risk PCa; and AUC_{phi+Fuc-PSA+SDC1+Tie-2}=0.936 or AUC_{phi+Fuc-PSA}=0.919 vs AUCₚₕᵢ=0.898, both p<0.0001 for AG vs low risk PCa and non-PCa).

The biomarker panels improved the specificity of AG PCa detection. For clinical applications, a very high sensitivity is required for the detection of AG PCa. In Table 2, to detect AG PCa from low risk PCa at a fixed sensitivity of 95.0%, the specificity of the four-marker panel of phi, Fuc-PSA, SDC1, and GDF-15, and the combination of phi and Fuc-PSA both had a specificity of 76.0% in comparison to that of 56.0% for phi (p=0.0²⁹, and 0.013, respectively) and 44.0% for PSA alone. Similarly, to detect AG PCa from low risk PCa and non-PCa at the same 95.0% sensitivity, the specificity for the four-marker panel of phi, Fuc-PSA, SDC1, and Tie-2, and the same phi, Fuc-PSA combinations had a specificity of 78.2% and 69.1%, respectively vs 65.5% for phi (p=0.010, and 0.207, respectively) and 36.4% for PSA alone.

### DISCUSSION

Serum PSA has been used as a sensitive marker for the detection of PCa, but it is not confined to PCa, elevated serum PSA levels have also been observed in benign prostatic hyperplasia (BPH) and prostatitis [27, 28]. Due to the potential for overdetection and overtreatment, PSA screening has caused controversy, posing a major challenge to the management of low-grade or low risk PCa [4]. Overdetection associated with PSA screening highlights the urgent need to identify more efficient biomarkers with improved specificity. Such novel biomarkers or sophisticated PSA derivative tests may address the clinical dilemma of differentiating AG from clinically indolent low risk PCa, and help physicians to select patients for biopsy. phi is one of tools approved by the FDA to improve the detection of PCa. Compared with PSA, phi, which incorporates PSA, p2PSA and fPSA in the equation, enhances the specificity of PCa detection [19, 29] and has also shown to be associated with AG PCa. In this study, consistent with our previous studies [7, 8], Fuc-PSA confirmed its ability to separate AG from either low risk PCa only or low risk PCa and non-PCa. Combining Fuc-PSA with phi improved the detection of AG PCa from either low risk PCa or low risk PCa and non-PCa, both with statistical significance in a bootstrap comparison of AUCs. Furthermore, two four-marker panels of phi, Fuc-PSA, SDC1, and GDF-15 or phi, Fuc-PSA, SDC1, and Tie-2 were identified with an even greater improved performance over phi individually to separate AG from either low risk PCa or low risk PCa and non-PCa with statistical significance. Clinically more relevant, compared with phi alone, the four-marker panels significantly improved the specificity of AG PCa detection. Improvement in specificity at a fixed 95% sensitivity was also observed comparing the combination of phi and Fuc-PSA with phi alone.

In this study, we further validated two serum biomarkers previously discovered from our EDRN BRL studies [6-8], Fuc-PSA and Tie-2, as effective biomarkers for the detection AG PCa either as individual biomarkers or used in combination with other biomarkers. In addition, we also demonstrated the potential diagnostic value of two serum biomarkers, SDC1 and GDF15, in two four-marker panels that separate AG from either low risk PCa only or low risk PCa and non-PCa patients. Compared with our previous studies [6-8], we expanded the evaluation of the diagnostic value of candidate biomarkers in detecting AG not only from low risk PCa only but also from low risk PCa and non-PCa cases. Our results, if validated in patient cohorts representative of intended populations, could have the potential as an in vitro diagnostic multivariate index assay (IVDMIA) to provide valuable clinical information to help detect AG PCa.

There are limitations to our analysis, as the sample size of the current study was not sufficient for separate independent evaluation. However, within this sample set, bootstrap resampling provides evidence of statistical stability of the observed improvement. Additional studies will be needed for validation and to test the generalizability of the improvement in performance in independent samples.

The results observed in this study are consistent with other reports showing that specific glycoforms of PSA can potentially be used as biomarkers, not only to improve the diagnostic accuracy of PCa, but also to detect AG tumors [30-33]. Changes in serum PSA sialylation have been reported in several studies [34-39], and specific increases in α2,3-sialic acid were observed in serum PSA in PCa patients compared with BPH and/or controls. In addition, increased core fucosylation of glycans has been detected in the serum of patients with PCa compared with healthy individuals or BPH [40, 41]. Previously, we developed multiplex immunoassays, based on AAL lectin affinity capturing and protein-antibody immunoreactivity, to analyze serum fucosylated glycoproteins in PCa patients [7]. Our data showed that Fuc-PSA was elevated and correlated with GS. Compared with total PSA, Fuc-PSA had better predictive ability to separate AG from low risk PCa. In addition, we previously developed two lectin-based immunoassays for the selection of glycoproteins containing fucosylated glycans using AAL and Lens culinaris agglutinin (LCA) followed by a clinical PSA immunoassay to analyze serum Fuc-PSA in PCa patients [8]. Our data suggested that Fuc-PSA-AAL, and Fuc-PSA-LCA levels may be effective biomarkers to separate AG [particularaly for GS≥7 (4+3)] from low risk PCa. AAL binds both core fucosylation and terminal fucosylation (α1-2/α1-3 fucosylation). In this study, we used agarose bound AAL beads to enrich Fuc-PSA from patient sera, therefore, the observed diagnostic value of serum Fuc-PSA in the detection of AG PCa could be attributed to both core fucosylation and terminal fucosylation of PSA, even though it has been reported that PSA fucosylation mainly occurs in the core glycan structure [42-44]. Contrary to these results, Llop, et al reported that the core fucosylation level of serum PSA in high-risk PCa was significantly reduced compared to BPH and low-risk PCa, with an Enzyme-linked Lectin Assay (ELLA) including a double immunoprecipitation of serum PSA followed by Phliota squarrosa lectin (PhoSL) detection, which recognizes only core fucosylation [38]. Contradictory reports on the glycosylation patterns of serum PSA may be attributed to a number of reasons. First, compared with antibodies, the binding affinity of lectins are much lower, and the concentration of PSA in patients' sera is very low, which makes the analysis of serum PSA glycosylation patterns very challenging, thus limiting the development of reliable assays with enough sensitivity for its detection in a large number of patient samples. Second, the analysis of serum PSA glycosylation patterns may be influenced by the glycosylated component present in complexed as opposed to free PSA forms. Lectins can bind not only to glycans on the target glycoproteins, but also to glycans on background glycoproteins (including antibodies), resulting in high background signals. Third, target and background glycoproteins might not be equally fucosylated, and multi-step sample preparation for glycan analysis could reduce quantitative accuracy and limit the analysis of a large number of patient samples in clinical studies to generate statistically significant data [31, 45].

Tie-2 is a transmembrane tyrosine kinase receptor for angiopoietins and plays a critical role in vascular development. It has been found to regulate the stemness and metastatic properties of PCa cells [17], and inhibiting angiopoietin-2 activity impedes angiogenesis and growth of LuCaP 23.1 PCa xenografts [16]. Our previous study showed that the soluble Tie-2 levels in sera of PCa patients with GS of 8-10 was significantly increased, indicating that Tie-2 shedding might be related to the aggressiveness of PCa [6].

SDC1 is one of four structurally related cell surface heparan sulfate proteoglycans and plays a pivotal role in cell-cell and cell-extracellular matrix interactions [46]. A significant increase in soluble SDC1 serum levels has been observed in advanced PCa cases, suggesting that SDC1 shedding might be related to PCa progression [47]. In addition, elevated serum SDC1 was shown to be an independent factor in adverse overall and disease-specific survival in a mutltivariable pre-operative model, making evaluation of serum SDC1 levels a promising tool for pre-operative risk-stratification and/or therapy monitoring.

GDF-15, also known as macrophage inhibitory cytokine 1 (MIC-1), is a member of the transforming growth factor beta (TGF-β) superfamily. It is synthesized as a 60-kDa dimer which is cleaved by furinlike proconvertases from its propeptide to release a 25-kDa mature protein [48]. Only processed mature GDF-15 diffuses into the circulation, while the unprocessed, propeptide-containing form is frequently secreted from tumor cells and remains localized in tissues due to strong matrix binding mediated by its propeptide [48]. Elevated serum GDF-15 levels have been found in many cancers, and shown to be a potentially valuable biomarker for cancer diagnosis and prognosis [25, 49, 50]. The diagnostic complementarity between serum GDF-15 and PSA and/or % fPSA in the detection of PCa from BPH has also been reported [49, 51, 52]. In this study, a significant increase in GDF-15 serum levels was observed in AG PCa cases compared with either low risk PCa or low risk PCa and non-PCa cases, which is consistent with the reports of elevated serum GDF15 in many cancers, including PCa [25, 49, 50]. Stephan et al, has found that the levels of serum GDF-15 in benign disease was higher than that in PCa [52], but increased serum GDF-15 concentration was strongly associated with advanced disease and progression of PCa [50]. Serum GDF-15 was found to be an independent marker of the presence of higher-grade (GS≥7) tumors, which was not solely due to tumor burden. This observation is likely due to differences in processed GDF-15 or changed extracellular matrix properties [52].

Although the serum Tie-2 and SDC1 levels in patients with AG PCa were found to be elevated as compared to those with low risk PCa or low risk PCa and non-PCa, these differences were not statistically significant, likely due to the limited sample size. A logistic regression model was constructed to evaluate the ability of Fuc-PSA to further improve performance of phi. We then identified other contributing factors including SDC1, GDF-15, and/or Tie-2, and further evaluated the diagnostic performance of serum biomarker combinations in separating AG from low risk PCa only or low risk PCa and non-PCa cases. Compared with phi and PSA analysis, the multivariate panels showed clinically meaningful improvements. The selection of optimal panels through multivariate logistic regression allowed us to identify markers that are complementary in detecting AG PCa. However, to use these panels of serum protein biomarkers clinically as an IVDMIA assay, additional large-scale independent validation studies of these panels combined with other clinical and analytical parameters will be required. Recently, there has been an increased interest in the detection of tumor-specific molecular alternations by high-throughput screening—“omic” technologies. There are many promising biomarkers, including various tumor and serum proteins, microRNAs, as well as genetic markers that may be combined as diagnostic or prognostic indices [53].

In conclusion, through systematic proteomics analysis of multivariate combinations of serum biomarkers, we have identified panels of biomarkers that are potentially capable of detecting AG PCa, and demonstrated clinically meaningful improvement on the diagnostic performance of phi. It would be valuable to validate these panels in a large cohort of patient samples, because confounding factors such as age, body mass index (BMI), diabetes, and race may also affect the results. The multivariate combinations of serum biomarkers identified in this study warrant further clinical validation in a different and larger patient population, which could contribute to the clinical management of prostate cancer.

### REFERENCES

1. Siegel R L, Miller K D, Fuchs H E, Jemal A. Cancer Statistics, 2021. C A Cancer J Clin. 2021; 71: 7-33.
2. Chan D W, Bruzek D J, Oesterling J E, Rock R C, Walsh P C. Prostate-specific antigen as a marker for prostatic cancer: a monoclonal and a polyclonal immunoassay compared. Clin Chem. 1987; 33: 1916-20.
3. USPSTF, Grossman D C, Curry S J, Owens D K, Bibbins-Domingo K, Caughey A B, et al. Screening for Prostate Cancer: U S Preventive Services Task Force Recommendation Statement. JAMA. 2018; 319: 1901-13.
4. Berman D M, Epstein J I. When is prostate cancer really cancer? Urol Clin North Am. 2014; 41: 339-46.
5. Pierorazio P M, Walsh P C, Partin A W, Epstein J I. Prognostic Gleason grade grouping: data based on the modified Gleason scoring system. BJU Int. 2013; 111: 753-60.
6. Li D, Chiu H, Gupta V, Chan D W. Validation of a multiplex immunoassay for serum angiogenic factors as biomarkers for aggressive prostate cancer. Clin Chim Acta. 2012; 413: 1506-11.
7. Li Q K, Chen L, Ao M H, Chiu J H, Zhang Z, Zhang H, et al. Serum fucosylated prostate-specific antigen (PSA) improves the differentiation of aggressive from non-aggressive prostate cancers. Theranostics. 2015; 5: 267-76.
8. Wang C, Hoti N, Lih T M, Sokoll L J, Zhang R, Zhang Z, et al. Development of a glycoproteomic strategy to detect more aggressive prostate cancer using lectin-immunoassays for serum fucosylated PSA. Clin Proteomics. 2019; 16: 13.
9. Hoti N, Lih T S, Pan J, Zhou Y, Yang G, Deng A, et al. A Comprehensive Analysis of FUT8 Overexpressing Prostate Cancer Cells Reveals the Role of EGFR in Castration Resistance. Cancers (Basel). 2020; 12.
10. Hoti N, Yang S, Hu Y, Shah P, Haffner M C, Zhang H. Overexpression of alpha (1,6) fucosyltransferase in the development of castration-resistant prostate cancer cells.

Prostate Cancer Prostatic Dis. 2018; 21: 137-46.
11. Wang X, Chen J, Li Q K, Peskoe S B, Zhang B, Choi C, et al. Overexpression of alpha (1,6) fucosyltransferase associated with aggressive prostate cancer. Glycobiology. 2014; 24: 935-44.
12. Li D, Satomura S. Biomarkers for Hepatocellular Carcinoma (HCC): An Update. Adv Exp Med Biol. 2015; 867: 179-93.
13. Li D, Mallory T, Satomura S. AFP-L3: a new generation of tumor marker for hepatocellular carcinoma. Clin Chim Acta. 2001; 313: 15-9.
14. Huss W J, Hanrahan C F, Barrios R J, Simons J W, Greenberg N M. Angiogenesis and prostate cancer: identification of a molecular progression switch. Cancer Res. 2001; 61: 2736-43.
15. Strohmeyer D, Rossing C, Strauss F, Bauerfeind A, Kaufmann O, Loening S. Tumor angiogenesis is associated with progression after radical prostatectomy in pT2/pT3 prostate cancer. Prostate. 2000; 42: 26-33.
16. Morrissey C, Dowell A, Koreckij T D, Nguyen H, Lakely B, Fanslow W C, et al. Inhibition of angiopoietin-2 in LuCaP 23.1 prostate cancer tumors decreases tumor growth and viability. Prostate. 2010; 70: 1799-808.
17. Tang K D, Holzapfel B M, Liu J, Lee T K, Ma S, Jovanovic L, et al. Tie-2 regulates the stemness and metastatic properties of prostate cancer cells. Oncotarget. 2016; 7: 2572-84.
18. Sokoll L J, Sanda M G, Feng Z, Kagan J, Mizrahi I A, Broyles D L, et al. A prospective, multicenter, National Cancer Institute Early Detection Research Network study of [−2]proPSA: improving prostate cancer detection and correlating with cancer aggressiveness. Cancer Epidemiol Biomarkers Prev. 2010; 19: 1193-200.
19. de la Calle C, Patil D, Wei J T, Scherr D S, Sokoll L, Chan D W, et al. Multicenter Evaluation of the Prostate Health Index to Detect Aggressive Prostate Cancer in Biopsy Naive Men. J Urol. 2015; 194: 65-72.
20. Alford A V, Brito J M, Yadav K K, Yadav S S, Tewari A K, Renzulli J. The Use of Biomarkers in Prostate Cancer Screening and Treatment. Rev Urol. 2017; 19: 221-34.
21. Sokoll L J, Wang Y, Feng Z, Kagan J, Partin A W, Sanda M G, et al. [−2]proenzyme prostate specific antigen for prostate cancer detection: a national cancer institute early detection research network validation study. J Urol. 2008; 180: 539-43; discussion 43.
22. Platt R W, Hanley J A, Yang H. Bootstrap confidence intervals for the sensitivity of a quantitative diagnostic test. Stat Med. 2000; 19: 313-22.
23. Qin G, Hsu Y S, Zhou X H. New confidence intervals for the difference between two sensitivities at a fixed level of specificity. Stat Med. 2006; 25: 3487-502.
24. Song J, Merbs S L, Sokoll L J, Chan D W, Zhang Z. A multiplex immunoassay of serum biomarkers for the detection of uveal melanoma. Clin Proteomics. 2019; 16: 10.
25. Song J, Sokoll L J, Pasay J J, Rubin A L, Li H, Bach D M, et al. Identification of Serum Biomarker Panels for the Early Detection of Pancreatic Cancer. Cancer Epidemiol Biomarkers Prev. 2019; 28: 174-82.
26. Gabriel K R. The Biplot Graphic Display of Matrices with Application to Principal Component Analysis. Biometrika. 1971; 58: 453-67.
27. Hasui Y, Marutsuka K, Asada Y, Ide H, Nishi S, Osada Y. Relationship between serum prostate specific antigen and histological prostatitis in patients with benign prostatic hyperplasia. Prostate. 1994; 25: 91-6.
28. Nadler R B, Humphrey P A, Smith D S, Catalona W J, Ratliff T L. Effect of inflammation and benign prostatic hyperplasia on elevated serum prostate specific antigen levels. J Urol. 1995; 154: 407-13.
29. Catalona W J, Partin A W, Sanda M G, Wei J T, Klee G G, Bangma C H, et al. A multicenter study of [−2]pro-prostate specific antigen combined with prostate specific antigen and free prostate specific antigen for prostate cancer detection in the 2.0 to 10.0 ng/ml prostate specific antigen range. J Urol. 2011; 185: 1650-5.
30. Munkley J, Mills I G, Elliott D J. The role of glycans in the development and progression of prostate cancer. Nat Rev Urol. 2016; 13: 324-33.
31. Vermassen T, Speeckaert M M, Lumen N, Rottey S, Delanghe J R. Glycosylation of prostate specific antigen and its potential diagnostic applications. Clin Chim Acta. 2012; 413: 1500-5.
32. Drake R R, Jones E E, Powers T W, Nyalwidhe J O. Altered glycosylation in prostate cancer. Adv Cancer Res. 2015; 126: 345-82.
33. Gilgunn S, Conroy P J, Saldova R, Rudd P M, O'Kennedy R J. Aberrant PSA glycosylation—a sweet predictor of prostate cancer. Nat Rev Urol. 2013; 10: 99-107.
34. Yoneyama T, Ohyama C, Hatakeyama S, Narita S, Habuchi T, Koie T, et al. Measurement of aberrant glycosylation of prostate specific antigen can improve specificity in early detection of prostate cancer. Biochem Biophys Res Commun. 2014; 448: 390-6.
35. Sarrats A, Saldova R, Comet J, O'Donoghue N, de Llorens R, Rudd P M, et al. Glycan characterization of PSA 2-D E subforms from serum and seminal plasma. OMICS. 2010; 14: 465-74.
36. Ohyama C, Hosono M, Nitta K, Oh-eda M, Yoshikawa K, Habuchi T, et al. Carbohydrate structure and differential binding of prostate specific antigen to Maackia amurensis lectin between prostate cancer and benign prostate hypertrophy. Glycobiology. 2004; 14: 671-9.
37. Meany D L, Zhang Z, Sokoll L J, Zhang H, Chan D W. Glycoproteomics for prostate cancer detection: changes in serum PSA glycosylation patterns. J Proteome Res. 2009; 8: 613-9.
38. Llop E, Ferrer-Batalle M, Barrabes S, Guerrero P E, Ramirez M, Saldova R, et al. Improvement of Prostate Cancer Diagnosis by Detecting PSA Glycosylation-Specific Changes. Theranostics. 2016; 6: 1190-204.
39. Ferrer-Batalle M, Llop E, Ramirez M, Aleixandre R N, Saez M, Comet J, et al. Comparative Study of Blood-Based Biomarkers, alpha2,3-Sialic Acid PSA and PHI, for High-Risk Prostate Cancer Detection. Int J Mol Sci. 2017; 18.
40. Kyselova Z, Mechref Y, Al Bataineh M M, Dobrolecki L E, Hickey R J, Vinson J, et al. Alterations in the serum glycome due to metastatic prostate cancer. J Proteome Res. 2007; 6: 1822-32.
41. Saldova R, Fan Y, Fitzpatrick J M, Watson R W, Rudd P M. Core fucosylation and alpha2-3 sialylation in serum N-glycome is significantly increased in prostate cancer comparing to benign prostate hyperplasia. Glycobiology. 2011; 21: 195-205.
42. Behnken H N, Ruthenbeck A, Schulz J M, Meyer B. Glycan analysis of Prostate Specific Antigen (PSA) directly from the intact glycoprotein by H R-ESI/TOF-M S. J Proteome Res. 2014; 13: 997-1001.
43. Tabares G, Radcliffe C M, Barrabes S, Ramirez M, Aleixandre R N, Hoesel W, et al. Different glycan structures in prostate-specific antigen from prostate cancer sera in relation to seminal plasma PSA. Glycobiology. 2006; 16: 132-45.
44. Peracaula R, Tabares G, Royle L, Harvey D J, Dwek R A, Rudd P M, et al. Altered glycosylation pattern allows the distinction between prostate-specific antigen (PSA) from normal and tumor origins. Glycobiology. 2003; 13: 457-70.
45. Li Y, Tian Y, Rezai T, Prakash A, Lopez M F, Chan D W, et al. Simultaneous analysis of glycosylated and sialylated prostate-specific antigen revealing differential distribution of glycosylated prostate-specific antigen isoforms in prostate cancer tissues. Anal Chem. 2011; 83: 240-5.
46. Gharbaran R. Advances in the molecular functions of syndecan-1 (SDC1/CD138) in the pathogenesis of malignancies. Crit Rev Oncol Hematol. 2015; 94: 1-17.
47. Szarvas T, Reis H, Vom Dorp F, Tschirdewahn S, Niedworok C, Nyirady P, et al. Soluble syndecan-1 (SDC1) serum level as an independent pre-operative predictor of cancer-specific survival in prostate cancer. Prostate. 2016; 76: 977-85.
48. Bauskin A R, Brown D A, Junankar S, Rasiah K K, Eggleton S, Hunter M, et al. The propeptide mediates formation of stromal stores of PROMIC-1: role in determining prostate cancer outcome. Cancer Res. 2005; 65: 2330-6.
49. Bansal N, Kumar D, Gupta A, Chandra D, Sankhwar S N, Mandhani A. Relevance of MIC-1 in the Era of PSA as a Serum Based Predictor of Prostate Cancer: A Critical Evaluation. Sci Rep. 2017; 7: 16824.
50. Brown D A, Lindmark F, Stattin P, Balter K, Adami H O, Zheng S L, et al. Macrophage inhibitory cytokine 1: a new prognostic marker in prostate cancer. Clin Cancer Res. 2009; 15: 6658-64.
51. Stephan C, Xu C, Brown D A, Breit S N, Michael A, Nakamura T, et al. Three new serum markers for prostate cancer detection within a percent free PSA-based artificial neural network. Prostate. 2006; 66: 651-9.
52. Brown D A, Stephan C, Ward R L, Law M, Hunter M, Bauskin A R, et al.

Measurement of serum levels of macrophage inhibitory cytokine 1 combined with prostate-specific antigen improves prostate cancer diagnosis. Clin Cancer Res. 2006; 12: 89-96.
53. Cohen J D, Li L, Wang Y, Thobum C, Afsari B, Danilova L, et al. Detection and localization of surgically resectable cancers with a multi-analyte blood test. Science. 2018; 359: 926-30.

TABLE 1
Clinicopathologic characteristics of the study cohort.

| | | Low Risk | AG | | AG PCa breakdown by GS | | | |
|---|---|---|---|---|---|---|---|---|
| | | PCa | PCa | | GS 7 | GS 7 | | |
| | Non-PCa | GS 6 | GS ≥ 7 | All PCa | (3 + 4) | (4 + 3) | GS 8 | GS 9 |
| Subjects, | 30 (33.7) | 30 (50.8) | 29 (49.2)^{§} | 59 (66.3)^{§} | 4 (6.8) | 6 (10.2) | 9 (15.3)^{§} | 10 (16.9) |
| n (%) | | | | | | | | |
| Age (y), | | | | | | | | |
| mean ± SD | 63.2 ± 8.6 | 61.3 ± 8.3 | 67.8 ± 9.9 | 64.5 ± 9.6 | 61.3 ± 7.6 | 66.7 ± 8.2 | 70.6 ± 11.4 | 68.5 ± 10.3 |
| (range) | (43.0-80.0) | (46.0-77.0) | (51.0-93.0) | (46.0-93.0) | (51.0-69.0) | (56.0-77.0) | (51.0-93.0) | (55.0-87.0) |
| Race, n (%) | | | | | | | | |
| White | 29 (96.7) | 25 (83.3) | 29 (100.0) | 54 (91.5) | 4 (100.0) | 6 (100.0) | 9 (100.0) | 10 (100.0) |
| Black | 0 (0.0) | 5 (16.7) | 0 (0.0) | 5 (8.5) | 0 (0.0) | 0 (0.0) | 0 (0.0) | 0 (0.0) |
| Asian | 1 (3.3) | 0 (0.0) | 0 (0.0) | 0 (0.0) | 0 (0.0) | 0 (0.0) | 0 (0.0) | 0 (0.0) |
| FHx of PCa, | | | | | | | | |
| n (%) | | | | | | | | |
| Yes | 8 (26.7) | 7 (23.3) | 11 (37.9) | 18 (30.5) | 3 (75.0) | 1 (16.7) | 3 (33.3) | 4 (40.0) |
| No | 22 (73.3) | 23 (76.7) | 18 (62.1) | 41 (69.55) | 1 (25.0) | 5 (83.3) | 6 (66.7) | 6 (60.0) |
| DRE, n (%), | | | | | | | | |
| Abnormal | 14 (46.7) | 4 (13.3) | 11 (37.9) | 15 (25.4) | 0 (0.0) | 3 (50.0) | 2 (22.2) | 6 (60.0) |
| Enlarged | 0 (0.0) | 6 (20.0) | 4 (13.8) | 10 (17.0) | 0 (0.0) | 0 (0.0) | 2 (22.2) | 2 (20.0) |
| Normal | 16 (53.3) | 20 (66.7) | 14 (48.3) | 34 (57.6) | 4 (100.0) | 3 (50.0) | 5 (55.6) | 2 (20.0) |
| Clinical | | | | | | | | |
| Stage (T) | | | | | | | | |
| T1c/x | n/a | 27/0 (90.0) | 20/1 (72.4) | 47/1 (81.4) | 3/1 (100.0) | 2/0 (33.3) | 7/0 (77.8) | 8/0 (80.0) |
| T2a/b/c/x | n/a | 2/0/0/0 (6.7) | 4/1/1/1 (24.1) | 6/1/1/1 (15.3) | 0/0/0/0 (0.0) | 2/1/0/1 (66.7) | 2/0/0/0 (22.2) | 0/0/1/0 (10.0) |
| T3a | n/a | 1 (3.3) | 1 (3.5) | 2 (3.3) | 0 (0.0) | 0 (0.0) | 0 (0.0) | 1 (10.0) |
| PSA | | | | | | | | |
| (ng/mL) | | | | | | | | |
| mean ± SD | 5.58 ± 3.59 | 4.43 ± 1.92* | 9.15 ± 5.84^{†, #} | 6.79 ± 4.92 | 7.43 ± 2.75 | 6.06 ± 2.11 | 12.48 ± 9.43^{†} | 9.00 ± 2.02^{#} |
| median | 4.97 | 4.58 | 7.98 | 5.79 | 6.67 | 5.99 | 11.35 | 9.17 |
| (range) | (0.47-18.44) | (0.41-9.01) | (0.85-30.98) | (0.41-30.98) | (5.14-11.24) | (3.59-9.70) | (0.85-30.98) | (6.24-12.14) |
| % fPSA | | | | | | | | |
| mean ± SD | 20.19 ± 9.81 | 20.54 ± 8.56* | 14.69 ± 6.50^{†, #} | 17.61 ± 8.08 | 15.91 ± 6.85 | 13.47 ± 5.89 | 16.00 ± 6.71^{†} | 13.69 ± 7.46^{#} |
| median | 17.7 | 18.56 | 15.68 | 17.1 | 16.25 | 13.26 | 16.4 | 12.57 |
| (range) | (5.66-43.80) | (8.64-51.34) | (3.63-25.52) | (3.63-51.34) | (7.21-23.93) | (6.34-21.46) | (5.34-23.86) | (3.63-25.52) |
| phi | | | | | | | | |
| mean ± SD | 26.52 ± 11.23 | 31.30 ± 15.32* | 66.19 ± 41.59^{†, #} | 48.74 ± 35.68 | 70.70 ± 49.18 | 52.35 ± 34.96 | 49.42 ± 9.61^{†} | 91.08 ± 54.49^{#} |
| median | 24.41 | 28.48 | 53.08 | 39.63 | 50.61 | 45.85 | 52.27 | 69.57 |
| (range) | (11.04-57.27) | (6.09-69.10) | (23.18-199.18) | (6.09-199.18) | (37.72-143.84) | (23.18-120.95) | (30.41-60.74) | (41.58-199.18) |

Note:
PCa, prostate cancer.
Non-PCa, biopsy negative.
AG, aggressive.
GS, Gleason score (biopsy).
FHx, family history.
DRE, digital rectal examination.
phi, prostate health index.
Median number of biopsy was 12 (range 8 to 20).
PCa in 4 cases with GS 6 was upgraded on prostatectomy pathology.
Original sample set n = 90, one problematic sample with a specimen quality issue.
was omitted in estimation of descriptive analysis (^{§}), additional missing data due to insufficient quantity for measurement were also indicated as *, ^{#}, and ^{†} for number of missing samples 4, 2, and 1, respectively.

TABLE 2
Biomarker panels improving the specificity of AG PCa detection.

| | AUC | | SP (%) | | | | |
|---|---|---|---|---|---|---|---|
| | (95% CI) | SN (%) | (p-value*) | True-Neg | True-Pos | False-Neg | False-Pos |
| AG vs Low Risk PCa | | | | | | | |
| Panel-1 | 0.942 (0.876-1.000) | 95.0 | 76.0 (0.029) | 19 | 24 | 1 | 6 |
| phi + Fuc-PSA | 0.914 (0.828-0.980) | 95.0 | 76.0 (0.013) | 19 | 24 | 1 | 6 |
| phi | 0.872 (0.748-0.971) | 95.0 | 56.0 | 14 | 24 | 1 | 11 |
| PSA | 0.866 (0.749-0.956) | 95.0 | 44.0 | 11 | 24 | 1 | 14 |
| AG vs Low Risk PCa | | | | | | | |
| & Non-PCa | | | | | | | |
| Panel-2 | 0.934 (0.866-0.987) | 95.0 | 78.2 (0.010) | 43 | 24 | 1 | 12 |
| phi + Fuc-PSA | 0.918 (0.842-0.974) | 95.0 | 69.1 (0.207) | 38 | 24 | 1 | 17 |
| phi | 0.898 (0.814-0.963) | 95.0 | 65.5 | 36 | 24 | 1 | 19 |
| PSA | 0.807 (0.697-0.905) | 95.0 | 36.4 | 20 | 24 | 1 | 35 |

Note:
PCa, prostate cancer.
AG, aggressive PCa.
Non-PCa, biopsy negative.
Panel-1, phi + Fuc-PSA + SDC1 + GDF-15.
Panel-2, phi + Fuc-PSA + SDC1 + Tie-2.
AUC, area under curve.
CI, confidence interval.
SN, sensitivity.
SP, specificity.
Neg, negative.
Pos, positive.
*, one-sided paired test comparing specificity against phi.

TABLE 3
Biomarker descriptive statistics.

| Biomarker | Subgroup | Number | Min | Max | Median | Mean | STD | IQR |
|---|---|---|---|---|---|---|---|---|
| B7-H3 | Non-PCa | 30 | 9.99 | 36.81 | 18.36 | 19.50 | 5.05 | 4.58 |
| | Low Risk PCa | 30 | 10.24 | 47.52 | 22.31 | 22.87 | 7.56 | 5.70 |
| | AG PCa | 29^{a} | 14.77 | 74.01 | 22.85 | 25.84 | 12.60 | 9.25 |
| PLA2G7 | Non-PCa | 30 | 22.68 | 139.06 | 81.81 | 81.72 | 31.90 | 42.36 |
| | Low Risk PCa | 30 | 24.87 | 163.22 | 84.32 | 94.21 | 38.96 | 64.88 |
| | AG PCa | 29^{a} | 50.49 | 149.22 | 100.23 | 101.59 | 25.97 | 48.26 |
| GDF-15 | Non-PCa | 30 | 0.17 | 1.88 | 0.89 | 0.98 | 0.43 | 0.66 |
| | Low Risk PCa | 30 | 0.35 | 4.40 | 0.89 | 1.17 | 0.89 | 0.55 |
| | AG PCa | 29^{a} | 0.58 | 4.92 | 1.19 | 1.57 | 1.10 | 0.83 |
| IL-6 R alpha | Non-PCa | 30 | 8.39 | 49.13 | 33.15 | 31.91 | 7.49 | 6.39 |
| | Low Risk PCa | 30 | 14.40 | 40.22 | 31.61 | 30.78 | 5.97 | 7.83 |
| | AG PCa | 29^{a} | 15.67 | 44.42 | 32.25 | 31.62 | 5.84 | 7.25 |
| SDC1 | Non-PCa | 30 | 1.62 | 4.77 | 2.59 | 2.75 | 0.70 | 1.02 |
| | Low Risk PCa | 30 | 1.81 | 3.54 | 2.54 | 2.58 | 0.52 | 0.76 |
| | AG PCa | 29^{a} | 1.90 | 5.40 | 2.76 | 2.87 | 0.77 | 0.91 |
| VCAM-1 | Non-PCa | 30 | 471.35 | 1641.00 | 894.76 | 866.64 | 286.83 | 414.75 |
| | Low Risk PCa | 30 | 406.97 | 1306.00 | 752.16 | 773.53 | 259.86 | 379.87 |
| | AG PCa | 29^{a} | 424.37 | 2251.80 | 908.17 | 927.48 | 403.58 | 419.38 |
| Tie-2 | Non-PCa | 30 | 5.27 | 33.29 | 15.54 | 14.98 | 4.85 | 5.48 |
| | Low Risk PCa | 30 | 7.12 | 34.95 | 13.89 | 14.83 | 5.76 | 5.98 |
| | AG PCa | 29^{a} | 7.10 | 39.54 | 16.00 | 16.29 | 5.72 | 5.30 |
| IL-16 | Non-PCa | 30 | 0.14 | 0.38 | 0.22 | 0.24 | 0.06 | 0.07 |
| | Low Risk PCa | 30 | 0.12 | 0.32 | 0.20 | 0.20 | 0.05 | 0.06 |
| | AG PCa | 29^{a} | 0.15 | 0.47 | 0.21 | 0.22 | 0.06 | 0.06 |
| CA15-3 | Non-PCa | 30 | 15.55 | 75.39 | 41.32 | 42.52 | 15.54 | 24.96 |
| | Low Risk PCa | 30 | 12.09 | 132.83 | 36.44 | 48.45 | 29.25 | 30.95 |
| | AG PCa | 29^{a} | 15.24 | 178.28 | 42.92 | 45.88 | 30.12 | 20.74 |
| MMP-2 | Non-PCa | 30 | 137.13 | 257.18 | 199.19 | 196.91 | 32.48 | 42.89 |
| | Low Risk PCa | 30 | 126.74 | 244.01 | 190.98 | 194.09 | 26.48 | 33.86 |
| | AG PCa | 29^{a} | 138.27 | 310.22 | 209.41 | 208.79 | 40.79 | 43.37 |
| HSP27 | Non-PCa | 30 | 0.12 | 2.27 | 0.37 | 0.45 | 0.43 | 0.23 |
| | Low Risk PCa | 30 | 0.16 | 1.77 | 0.42 | 0.51 | 0.39 | 0.37 |
| | AG PCa | 29^{a} | 0.14 | 1.20 | 0.41 | 0.49 | 0.30 | 0.41 |
| Fuc-PSA* | Non-PCa | 30 | 0.05 | 1.14 | 0.23 | 0.30 | 0.23 | 0.22 |
| | Low Risk PCa | 25^{b} | 0.03 | 0.45 | 0.15 | 0.19 | 0.12 | 0.12 |
| | AG PCa | 25^{a, b} | 0.16 | 1.99 | 0.30 | 0.54 | 0.47 | 0.41 |
| PSA | Non-PCa | 30 | 0.47 | 18.44 | 4.97 | 5.58 | 3.59 | 4.26 |
| | Low Risk PCa | 26^{b} | 0.41 | 9.01 | 4.58 | 4.43 | 1.92 | 2.76 |
| | AG PCa | 26^{a, b} | 0.85 | 30.98 | 7.98 | 9.15 | 5.84 | 4.34 |
| % fPSA | Non-PCa | 30 | 5.66 | 43.80 | 17.70 | 20.19 | 9.81 | 15.16 |
| | Low Risk PCa | 26^{b} | 8.64 | 51.34 | 18.56 | 20.54 | 8.56 | 7.41 |
| | AG PCa | 26^{a, b} | 3.63 | 25.52 | 15.68 | 14.69 | 6.50 | 11.04 |
| phi | Non-PCa | 30 | 11.04 | 57.27 | 24.41 | 26.52 | 11.23 | 13.83 |
| | Low Risk PCa | 26^{b} | 6.09 | 69.10 | 28.48 | 31.30 | 15.32 | 11.63 |
| | AG PCa | 26^{a, b} | 23.18 | 199.18 | 53.08 | 66.19 | 41.59 | 14.90 |

1. A method for identifying a patient as having aggressive prostate cancer comprising the steps of:
(a) measuring the concentration of total PSA, free PSA, p2PSA in a serum sample obtained from the patient and calculating phi based on the measured serum concentrations;
(b) measuring the concentration of fucosylated PSA (fuc-PSA) in a serum sample obtained from the patient;
(c) measuring the concentration in a serum sample obtained from the patient of one or more of the following biomarkers: B7-H3, PLA2G7, GDF-15, IL-6 R alpha, SDC1, VCAM-1, sTie-2, IL-16, CA15-3, MMP-2, and HSP27; and
(d) using an algorithm to identify the patient as having aggressive prostate cancer based on a panel of biomarkers comprising phi, fuc-PSA and one or more of the serum concentrations measured in step (c).

2. The method of claim 1, wherein the panel of step (d) comprises phi, fuc-PSA, SDC1 and GDF-15.

3. The method of claim 1, wherein the panel of step (d) comprises phi, fuc-PSA, SDC1 and Tie-2.

4. The method of claim 1, wherein the panel of step (d) further comprises PSA and % fuc-PSA.

5. The method of claim 1, wherein measurement steps (a) and (c) are performed using an immunoassay.

6. The method of claim 1, wherein measurement step (b) is performed using a lectin assay followed by an immunoassay.

7. The method of claim 1, further comprising the step of treating the patient with a prostate cancer therapy.

8. The method of claim 7, wherein the prostate cancer therapy comprises prostatectomy, radiation therapy, cryotherapy, hormone therapy, chemotherapy, immunotherapy and combinations thereof.

9. The method of claim 1, wherein the panel comprises phi, fuc-PSA, SDC1, GDF-15, IL-6 R alpha, MMP-2 and CA15-3.

10. The method of claim 1, wherein the panel comprises phi, fuc-PSA and PLA2G7.

11. The method of claim 4, wherein the panel comprises phi, fuc-PSA, PSA, % fuc-PSA and GDF-15.

12. The method of claim 4, wherein the panel comprises phi, fuc-PSA, PSA, % fuc-PSA and B7-H3.

13. The method of claim 4, wherein the panel comprises phi, fuc-PSA, PSA, % fuc-PSA, GDF-15, SDC1, Tie-2 and VCAM-1.

14. The method of claim 4, wherein the panel comprises phi, fuc-PSA, PSA, % fuc-PSA, GDF-15, B7-H3, Tie-2, and SDC1.

15. A method for identifying a patient as having aggressive prostate cancer comprising the steps of:
(a) measuring the concentration of total PSA, free PSA, p2PSA in a serum sample obtained from the patient and calculating phi based on the measured serum concentrations;
(b) measuring the concentration of fucosylated PSA (fuc-PSA) in a serum sample obtained from the patient; and
(c) using an algorithm to identify the patient as having aggressive prostate cancer based on a panel of biomarkers comprising phi and fuc-PSA.

16. The method of claim 15, wherein measurement step (a) is performed using an immunoassay.

17. The method of claim 15, wherein measurement step (b) is performed using a lectin assay followed by an immunoassay.

18. The method of claim 15, further comprising the step of treating the patient with a prostate cancer therapy.

19. The method of claim 16, wherein the prostate cancer therapy comprises prostatectomy, radiation therapy, cryotherapy, hormone therapy, chemotherapy, immunotherapy and combinations thereof.

20. A method for treating a patient having aggressive prostate cancer comprising the step of administering a prostate cancer therapy to a patient identified as having aggressive prostate cancer using the method of claim 1.
